(19) United States
(12) Patent Application Publication
(10) Pub. No.: US 2025/0295653 A1
(43) Pub. Date: Sep. 25, 2025

(54) Use Of Glycine Transporter- 1 Antagonists As Vascular Dementia And/Or Stroke Prophylactic/Treatment Agents

(71) Applicant: Recherches Neuro-Hippocampe Inc., Ottawa (CA)

(72) Inventor: Richard Bergeron, Ottawa (CA)

(21) Appl. No.: 18/863,514

(22) PCT Filed: Nov. 28, 2022
(86) PCT No.: PCT/CA2022/000057

§ 371 Date: Nov. 6, 2024

(30) Foreign Application Priority Data
May 6, 2022 (CA) 3157880
May 6, 2022 (CA) 3157907

(51) Int. Cl. A61K 31/496; A61K 31/198; A61K 31/4178; A61K 31/422; A61P 9/10; A61P 25/28

(52) U.S. Cl. CPC A61K 31/496; A61K 31/198; A61K 31/4178; A61K 31/422; A61P 9/10; A61P 25/28

(57) ABSTRACT

A use of a compound of the class of glycine transporter-1 antagonist compounds as a vascular dementia prophylactic/treatment agent is provided. Furthermore, a use of a compound of the class of glycine transporter-1 antagonist compounds as a stroke prophylactic/treatment agent is provided.

### FIELD OF THE INVENTION

The present invention relates to glycine transporter-1 antagonists, and more particularly to the use of glycine transporter-1 antagonists as vascular dementia and/or stroke prophylactic/treatment agents.

### BACKGROUND OF THE INVENTION

Strokes are caused by a change in blood supply to a particular area of the brain. Most commonly, a blood clot will block blood flow to a particular blood vessel or a particular group of blood vessels in what is called an ischemic stroke. Vascular dementia is the second most common form of dementia after Alzheimer's disease. Vascular dementia is caused by problems in the supply of blood to the brain, typically in the form of a series of minor strokes, leading to worsening cognitive abilities. On average, people with vascular dementia live for around five years after symptoms begin, less than the average for Alzheimer's disease. Stroke and vascular dementia share many of the same risk factors as heart attack such as, for example, age, hypertension, smoking, hypercholesterolemia, diabetes, cardiovascular disease, and cerebrovascular disease.

Tissue Plasminogen Activator (tPA) “Alteplase” is the current gold-standard for acute stroke treatment. Although it works well, it must be administered within 3-4 hrs of symptom onset and can cause hemorrhaging as a potentially lethal side effect, resulting in only a few patients being eligible to receive this treatment.

Tenecteplase is a newer version of Alteplase, but has the same short therapeutic window and the same potentially lethal side effects, since it belongs to the same class of medication as Alteplase.

Acetylsalicylic acid (Aspirin®) is currently prescribed at a low dose (usually 81 mg) prophylactically to patients who are at risk for stroke. Although effective, long term use has been associated to severe irritation of the inner lining of the stomach.

It is desirable to provide a prophylactic/treatment agent capable of substantially reducing the severity of vascular dementia and/or stroke.

It is also desirable to provide a prophylactic agent capable of substantially reducing the severity of vascular dementia and/or stroke that has no known severe side effects related to long term use.

It is also desirable to provide a treatment agent capable of treating vascular dementia and/or stroke that has no known severe side effects.

It is also desirable to provide a prophylactic agent capable of substantially reducing the severity of vascular dementia and/or stroke that is not thrombolytic and therefore does not carry a severe risk of bleeding.

It is also desirable to provide a treatment agent capable of treating vascular dementia and/or stroke that is not thrombolytic and therefore does not carry a severe risk of bleeding.

### SUMMARY OF THE INVENTION

Accordingly, one object of the present invention is to provide a prophylactic agent capable of substantially reducing the severity of vascular dementia and/or stroke.

Another object of the present invention is to provide a prophylactic agent capable of substantially reducing the severity of vascular dementia and/or stroke that has no known severe side effects related to long term use.

Another object of the present invention is to provide a treatment agent capable of treating vascular dementia and/or stroke that has no known severe side effects.

Another object of the present invention is to provide a prophylactic agent capable of substantially reducing the severity of vascular dementia and/or stroke that is not thrombolytic and therefore does not carry a severe risk of bleeding.

Another object of the present invention is to provide a treatment agent capable of treating vascular dementia and/or stroke that is not thrombolytic and therefore does not carry a severe risk of bleeding.

According to one aspect of the present invention, there is provided a use of a compound of the class of glycine transporter-1 antagonist compounds as a vascular dementia prophylactic agent.

According to the aspect of the present invention, there is provided a use of a compound of the class of glycine transporter-1 antagonist compounds as a stroke prophylactic agent.

According to another aspect of the present invention, there is provided a use of a compound of the class of glycine transporter-1 antagonist compounds as a vascular dementia treatment agent.

According to the other aspect of the present invention, there is provided a use of a compound of the class of glycine transporter-1 antagonist compounds as a stroke treatment agent.

The advantage of the present invention is that it provides a prophylactic agent capable of substantially reducing the severity of vascular dementia and/or stroke.

A further advantage of the present invention is that it provides a prophylactic agent capable of substantially reducing the severity of vascular dementia and/or stroke that has no known severe side effects related to long term use.

A further advantage of the present invention is that it provides a treatment agent capable of treating vascular dementia and/or stroke that has no known severe side effects.

A further advantage of the present invention is that it provides a prophylactic agent capable of substantially reducing the severity of vascular dementia and/or stroke that is not thrombolytic and therefore does not carry a severe risk of bleeding.

A further advantage of the present invention is that it provides a treatment agent capable of treating vascular dementia and/or stroke that is not thrombolytic and therefore does not carry a severe risk of bleeding.

### BRIEF DESCRIPTION OF THE DRAWINGS

A preferred embodiment of the present invention is described below with reference to the accompanying drawings, in which:

FIG. 1a is a diagram illustrating normalized raw traces showing the effect of increasing concentrations of exogenous glycine on Schaffer Collateral NMDAR-EPSCs and mean time course data showing the effect of a 15 mins application of various glycine concentrations of pre-clinical tests as described herein according to the invention;

FIG. 1b is a diagram illustrating a dose-response curve of glycine and NMDAR-EPSC amplitudes of pre-clinical tests as described herein according to the invention;

FIG. 1c is a diagram illustrating the effect of 250 μM and 1 mM glycine on NMDAR-EPSC amplitudes in the presence of a dynasore of pre-clinical tests as described herein according to the invention;

FIG. 1d is a diagram illustrating the role of various concentrations of extracellular Ca²⁺ on NMDAR-EPSC amplitudes in the presence of 1 mM glycine, as well as changing intracellular Ca²⁺ levels using BAPTA, nimodipine, or CPA of pre-clinical tests as described herein according to the invention;

FIG. 1e is a diagram illustrating the effect of various concentrations of glycine on NMDAR-EPSC amplitudes in GlyT1^{+/-} mice, compared to WT of pre-clinical tests as described herein according to the invention;

FIG. 1f is a diagram illustrating the dose-response curve of the effects of glycine on NMDAR amplitudes in GlyT1^{+/-} mice of pre-clinical tests as described herein according to the invention;

FIG. 1g is a diagram illustrating the effect of 250 μM glycine on NMDAR-EPSC amplitudes in GlyT1^{+/-} mice in the presence of dynasore of pre-clinical tests as described herein according to the invention;

FIG. 1h is a diagram illustrating the effect of low (10 μM) and high (1 mM) concentrations of glycine or D-serine concentrations on NMDAR-EPSC amplitudes of pre-clinical tests as described herein according to the invention;

FIG. 1i is a diagram illustrating the dose-response curve of NMDAR-EPSC amplitudes to D-serine compared to glycine of pre-clinical tests as described herein according to the invention;

FIG. 1j is a diagram illustrating the effect of 1 mM D-serine in the presence of dynasore of pre-clinical tests as described herein according to the invention;

FIG. 1k is a diagram illustrating the effect of 10 UM D-serine while elevating endogenous glycine levels with NFPS of pre-clinical tests as described herein according to the invention;

FIG. 11 is a diagram illustrating a dose-response curve showing the effect of exogenous D-serine levels on SR-mice of pre-clinical tests as described herein according to the invention;

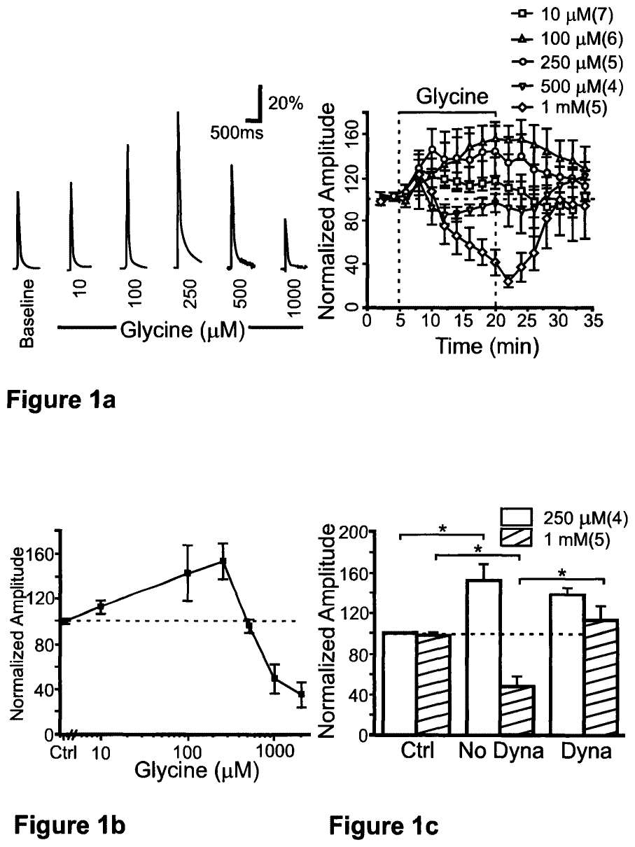

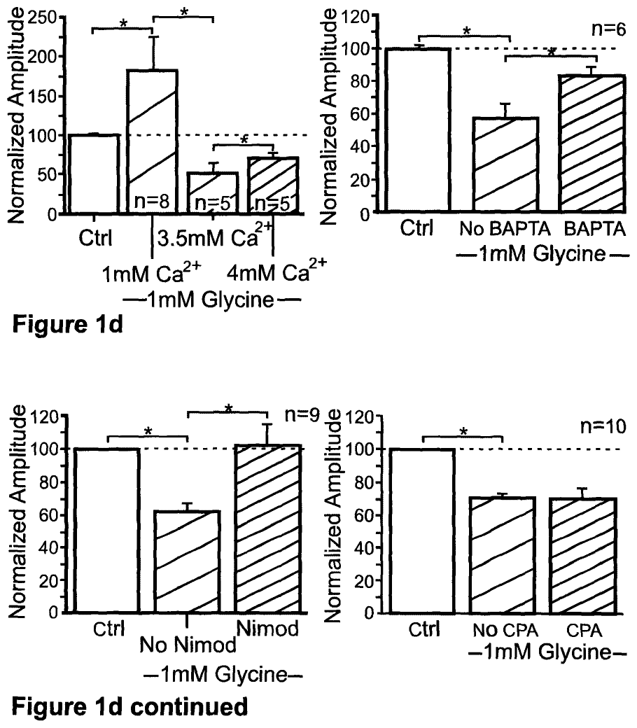

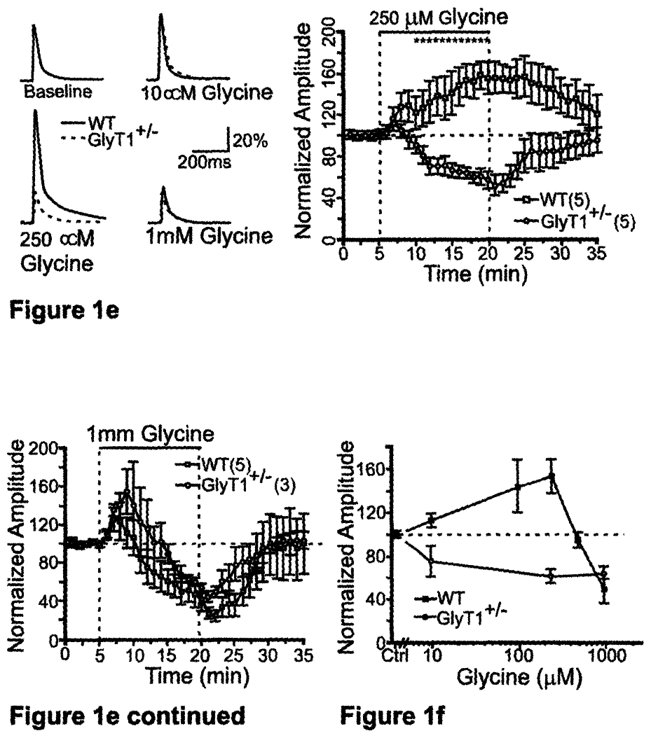

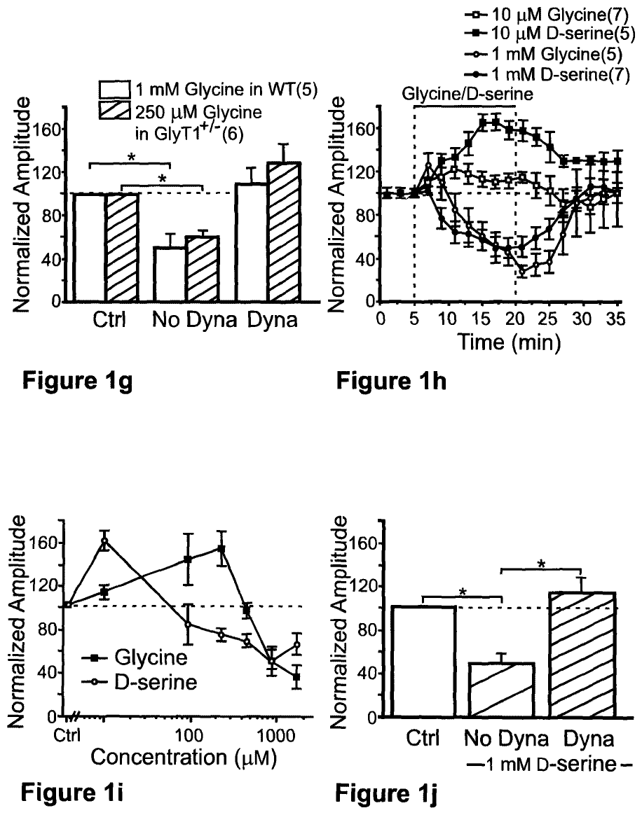

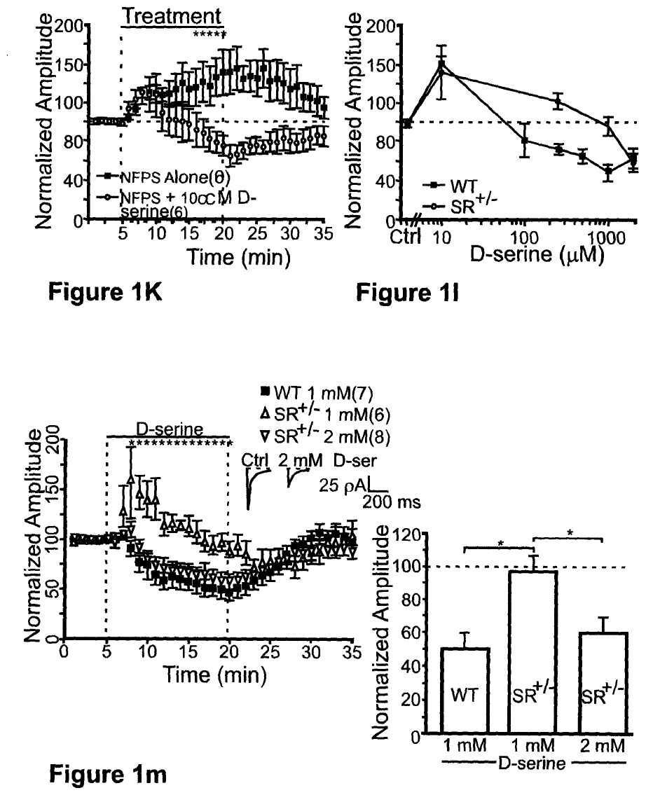

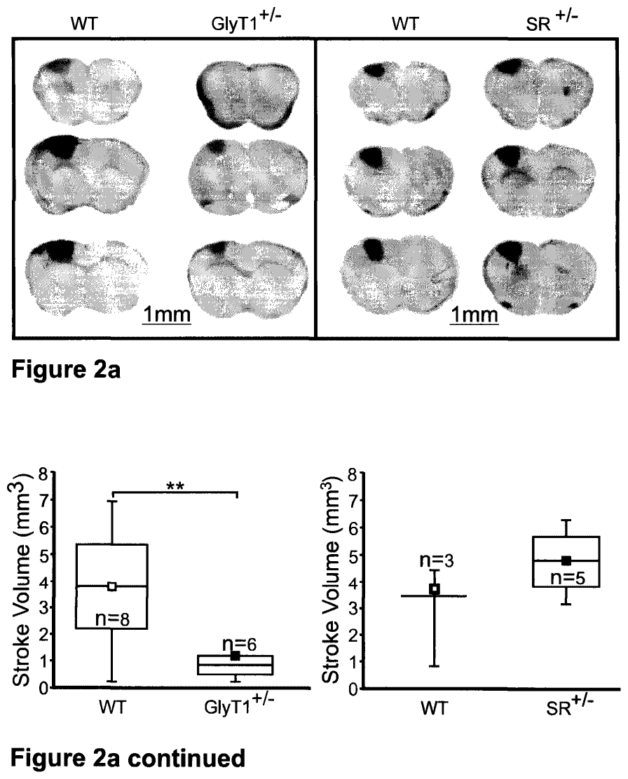

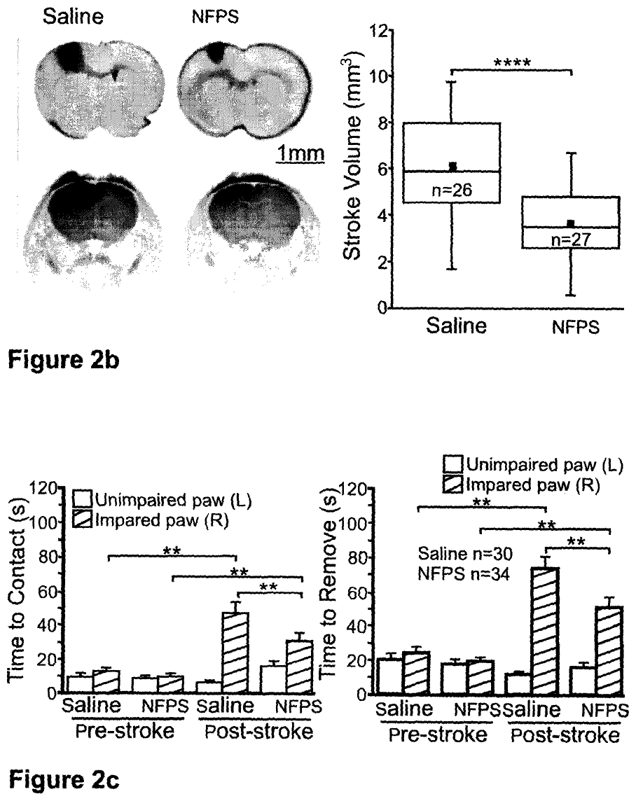

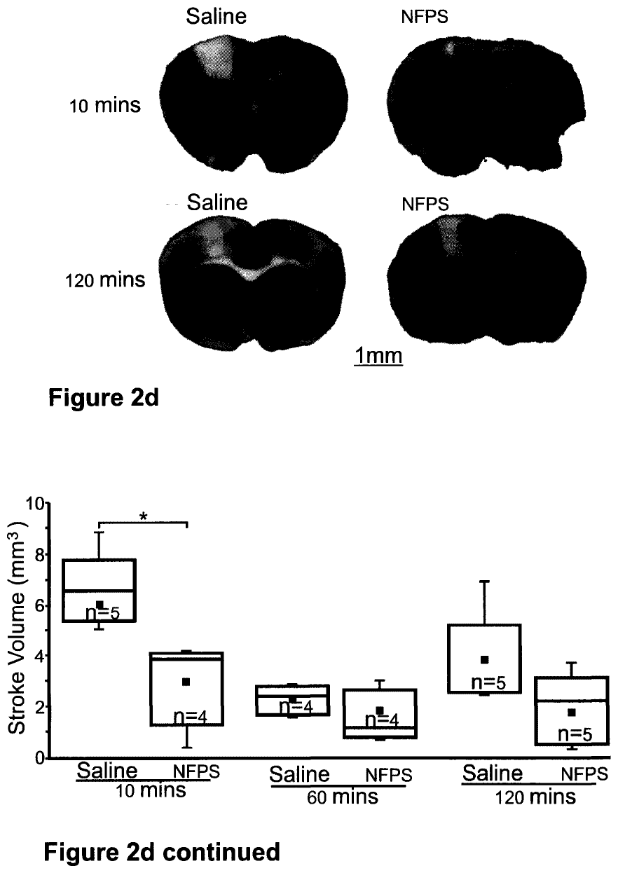

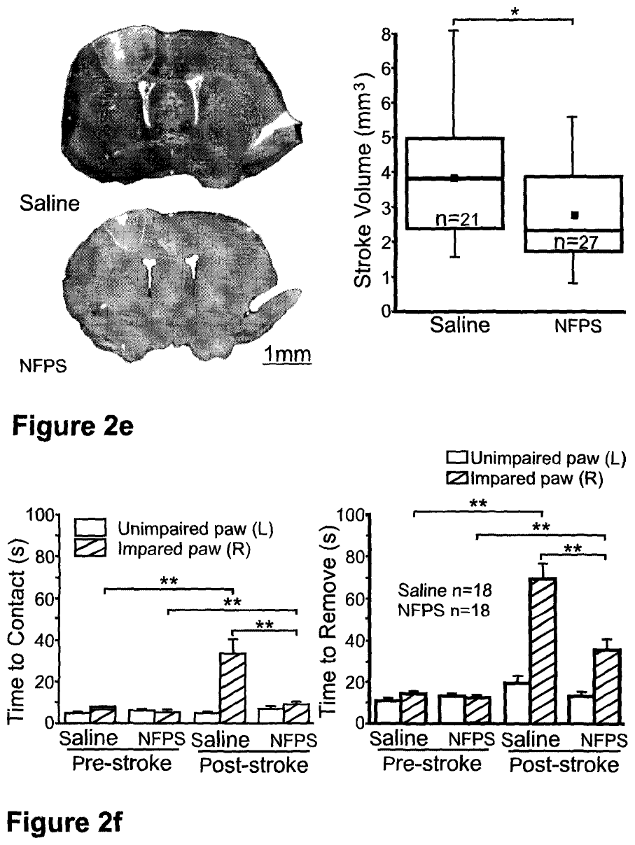

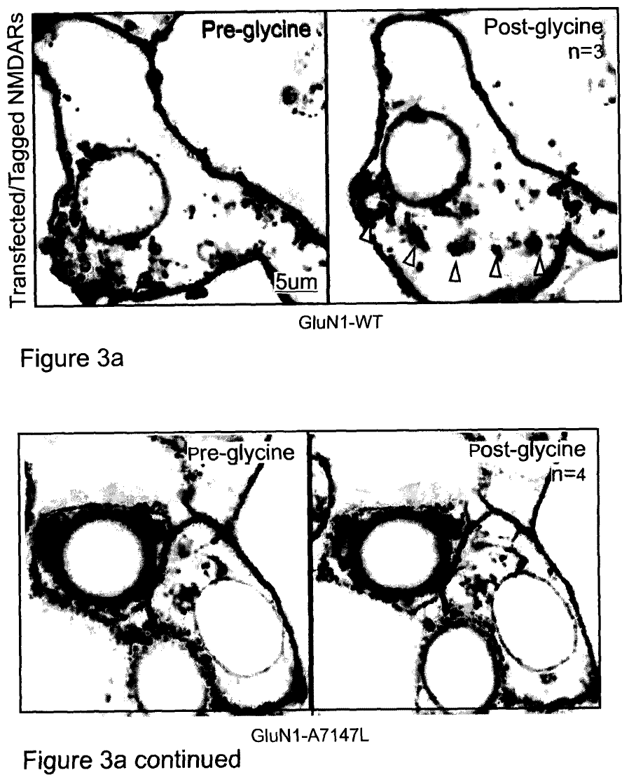

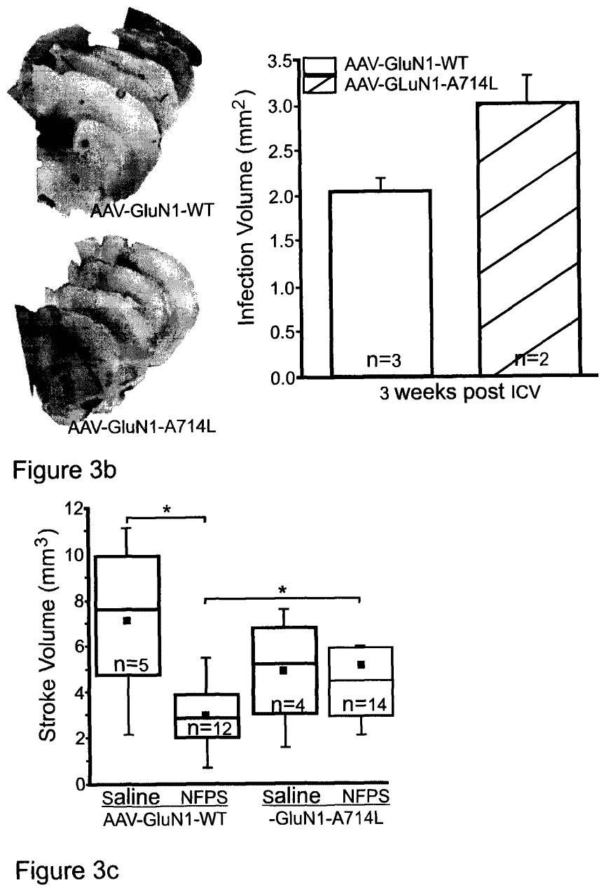

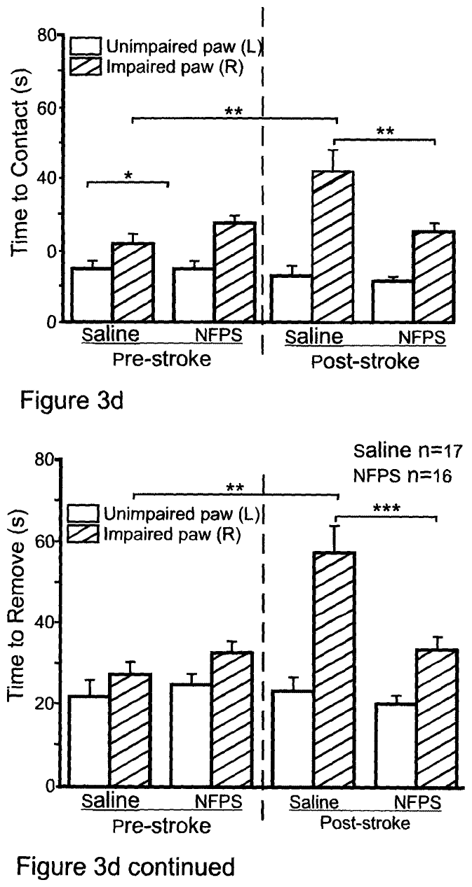

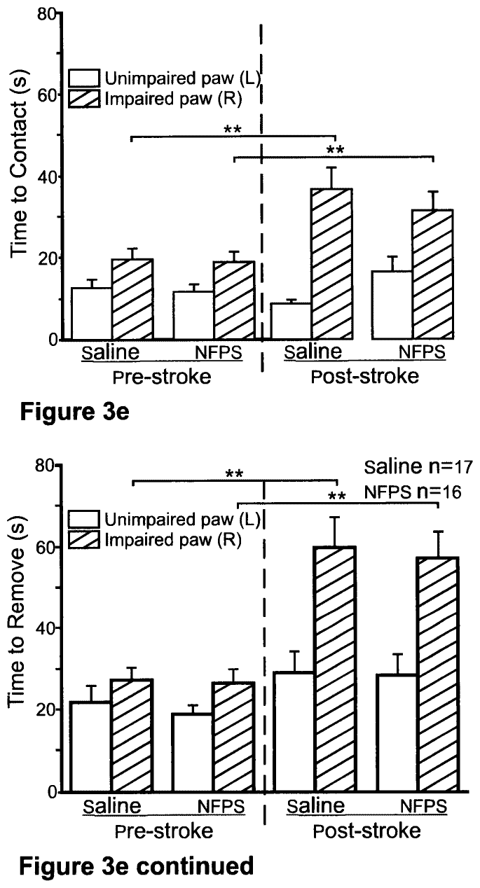

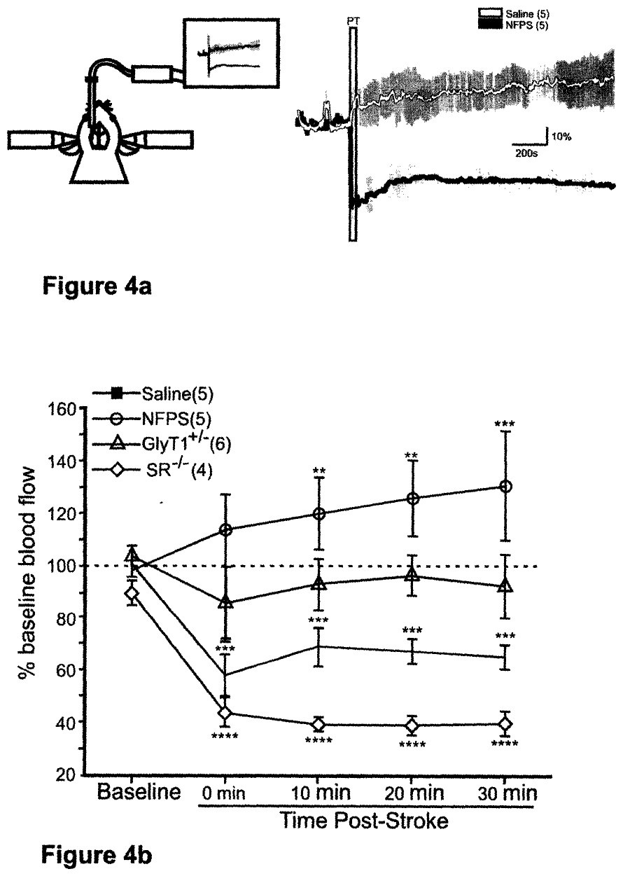

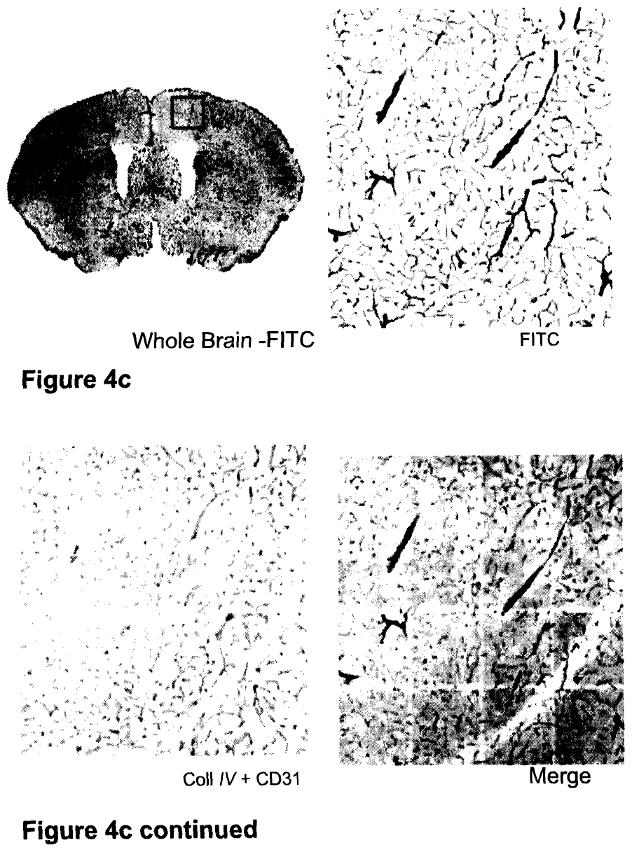

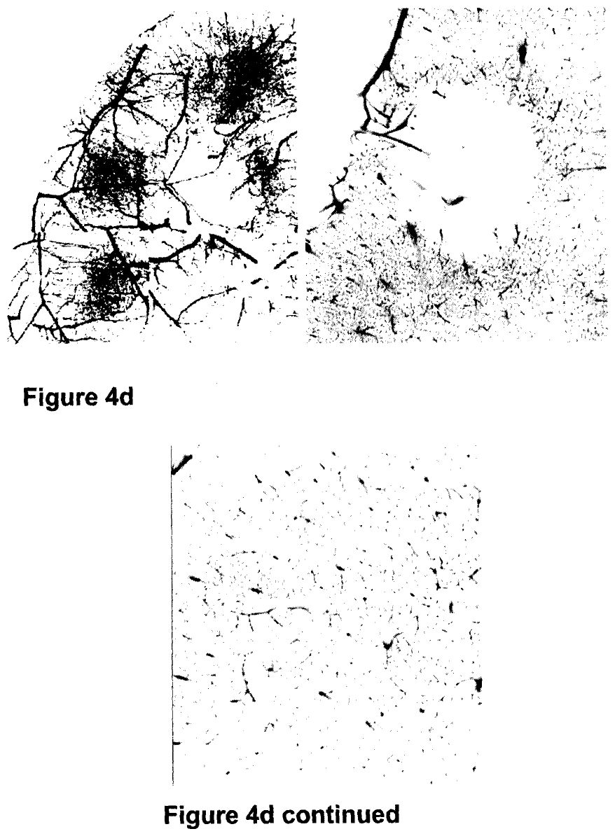

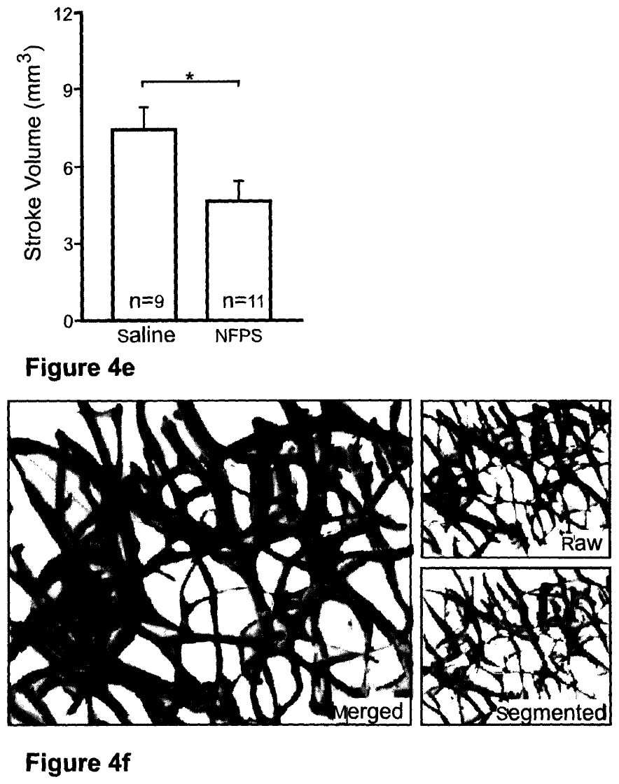

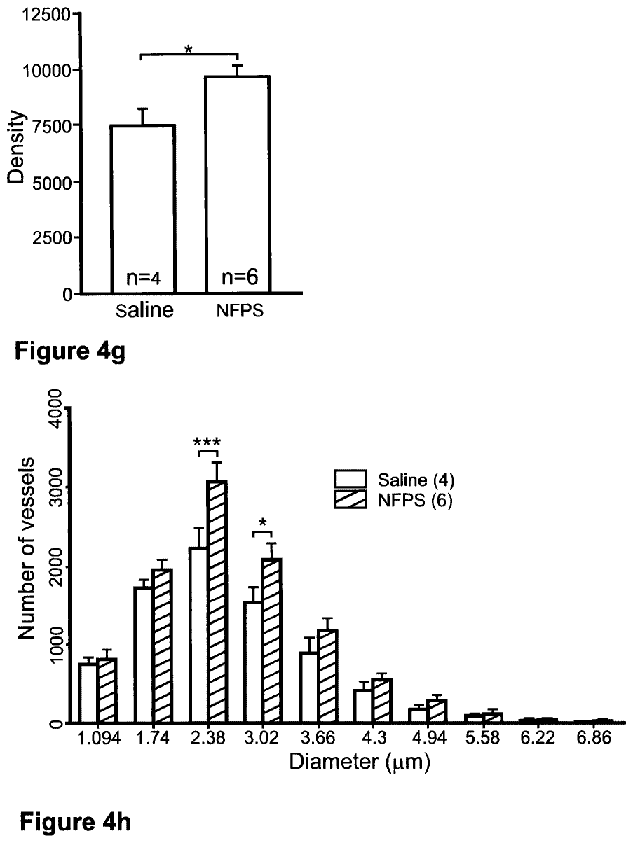

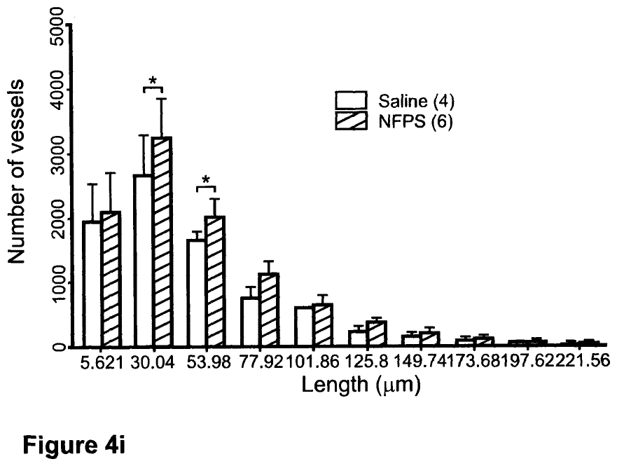

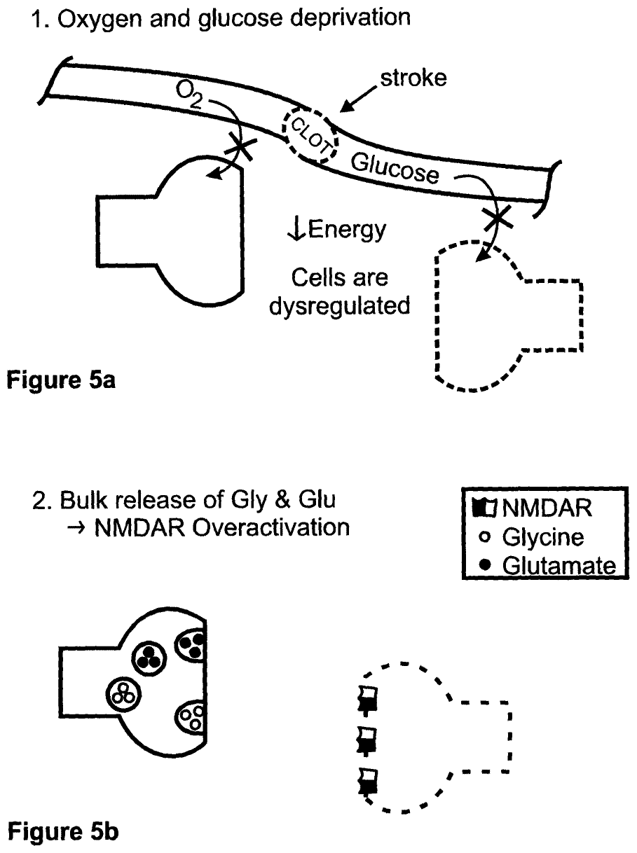

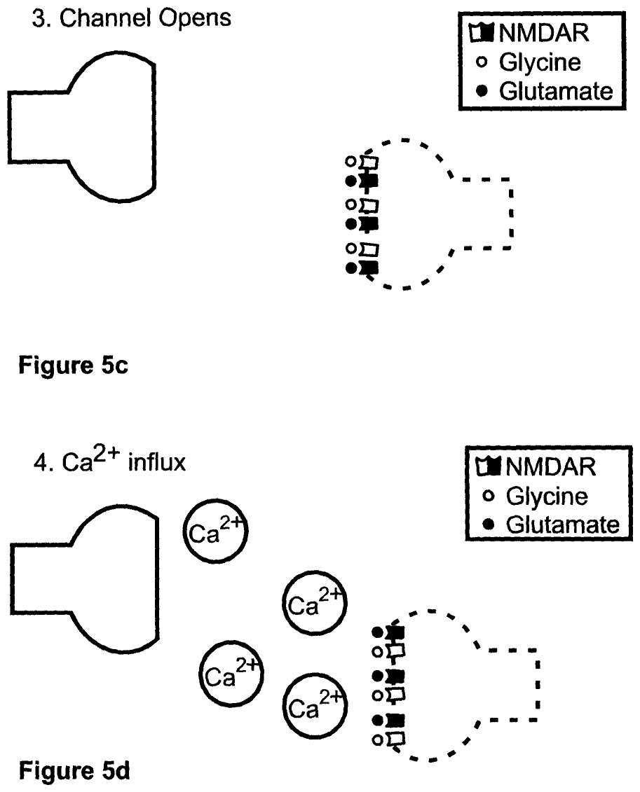

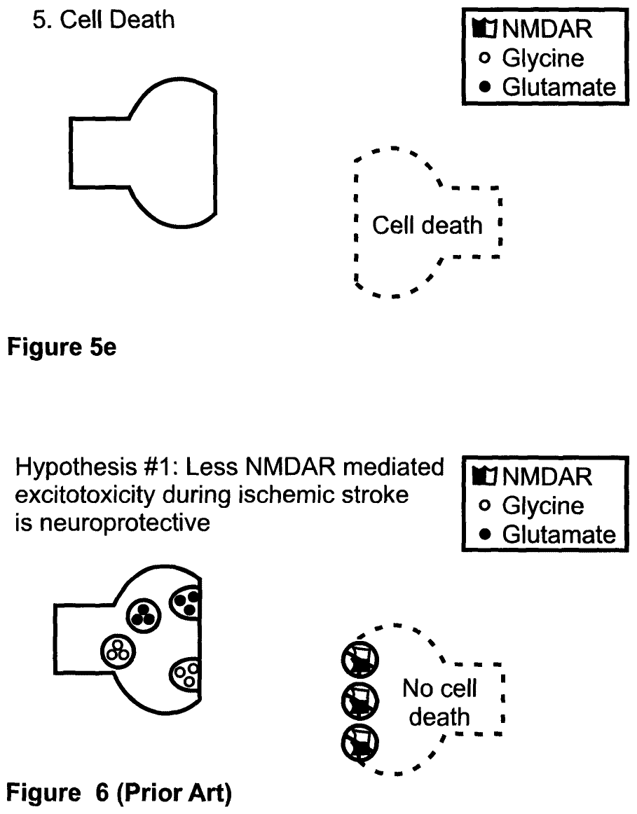

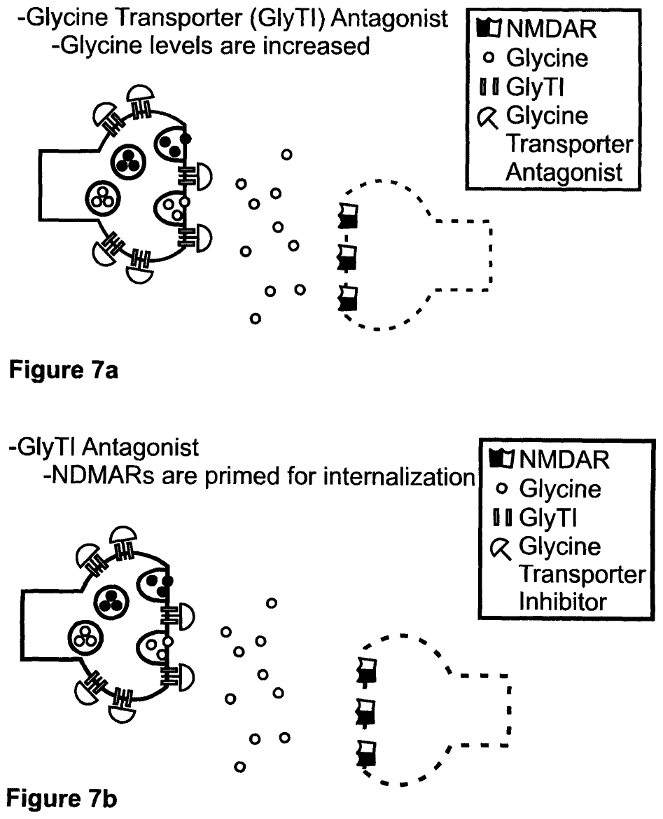

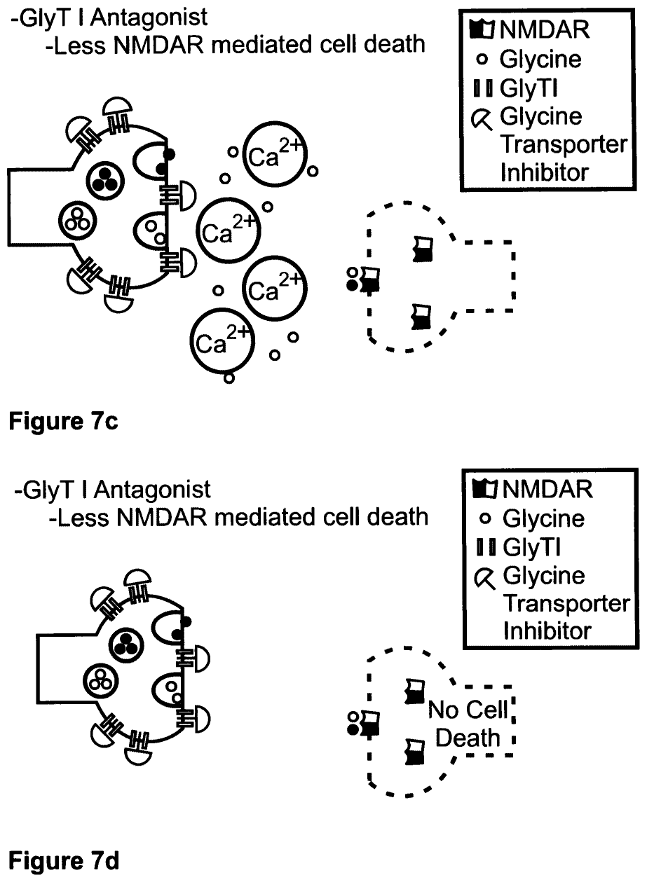

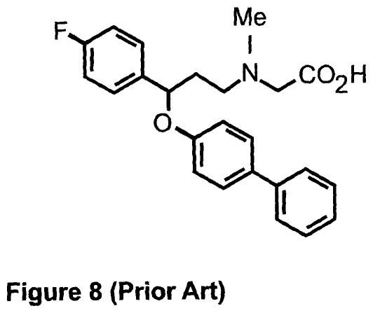

FIG. 1m is a diagram illustrating the effect of a higher dose of D-serine (1 mM vs. 2 mM) on NMDAR-EPSC amplitudes in SR^{-/-} mice compared to their WT littermates. Data is mean±SEM; statistical significance p<0.05 of pre-clinical tests as described herein according to the invention;

FIG. 2a is a diagram illustrating representative serial coronal sections of TTC-stained mouse forebrain (slice thickness 500 μM) and their corresponding box and whisker plots showing the infarct volume when assessed 48 hrs after the induction of a unilateral photothrombotic stroke in GlyT1^{+/-} and SR/mice relative to WT mice of pre-clinical tests as described herein according to the invention;

FIG. 2b is a diagram illustrating representative TTC-stained (top) or magnetic resonance imaging (bottom) sections showing representative stroke regions observed 48 hrs following the induction of photothrombotic (PT) stroke, and a box and whisker plot showing stroke volume in saline-treated or NFPS-treated mice 24 hrs before stroke induction of pre-clinical tests as described herein according to the invention;

FIG. 2c is a diagram illustrating the effect of 24 hrs pre-stroke NFPS administration on post-stroke time to contact and time to remove in the adhesive removal task compared with saline treatment, when evaluated 48 hrs following PT stroke of pre-clinical tests as described herein according to the invention;

FIG. 2d is a diagram illustrating effect of various post-stroke administration time-points of NFPS treatment on stroke volume with their corresponding box and whisker plots following PT stroke of pre-clinical tests as described herein according to the invention;

FIG. 2e is a diagram illustrating representative cresyl violet sections (25 μm thick) 48 hrs following endothelin-1 (ET-1) stroke obtained from saline-treated and NFPS-treated mice 24 hrs prior, in which the extent of the infarct is shown within the yellow border and box and whisker plot depicting infarct volume of pre-clinical tests as described herein according to the invention;

FIG. 2f is a diagram illustrating the effect of 24 hrs pre-stroke NFPS administration on post-stroke time to contact and time to remove in the adhesive removal task compared with saline treatment following ET-1 stroke. Data is mean±SEM; statistical significance p<0.05*, p<0.01**, p<0.001***, and p<0.0001**** of pre-clinical tests as described herein according to the invention;

FIG. 3a is a diagram illustrating visual representation of NMDAR internalization in GluN1-WT or GluN1-A714L transfected HEK293 cells following application of 1 mM glycine. Transfected NMDARs are labeled in green, whereas extracellular NMDARs are additionally labeled with red cell impermeable nanobody staining of pre-clinical tests as described herein according to the invention;

FIG. 3b is a diagram illustrating representative images showing the extent of viral spread in the mouse forebrain following infection between mice infected with AAV-GluN1-WT or AAV-GluN1-A714L of pre-clinical tests as described herein according to the invention;

FIG. 3c is a diagram illustrating box and whisker plot showing the effect of NFPS administration 24 hrs before PT stroke induction in mice infected with AAV-GluN1-WT or AAV-GluN1-A714L of pre-clinical tests as described herein according to the invention;

FIGS. 3d and 3e are diagrams illustrating the effect of NFPS on post-stroke time to contact and time to remove in the adhesive removal task compared with saline treatment, in mice infected with AAV-GluN1-WT (D), and in mice infected with AAV-GluN1-A714L (E) 48 hrs following PT stroke. Data is mean±SEM; statistical significance p<0.05*, p<0.01**, and p<0.001*** of pre-clinical tests as described herein according to the invention;

FIG. 4a is a diagram illustrating laser doppler flowmetry set-up and the measured effect of NFPS on cerebral blood flow following photothrombotic (PT) stroke of pre-clinical tests as described herein according to the invention;

FIG. 4b is a diagram illustrating time course of post-stroke cerebral blood flow following PT in mice with varying levels of glycine or D-serine of pre-clinical tests as described herein according to the invention;

FIG. 4c is a diagram illustrating 50 μm coronal section of brain perfused with FITC-BSA. Magnified images from the sensorimotor cortex demonstrating exact colocalization of FITC-BSA perfusion (green) with CD31 and CollIV vascular immunostaining (purple) of pre-clinical tests as described herein according to the invention;

FIG. 4d is a diagram illustrating colorized max projection of stroked hemisphere, and single section of raw images depicting vasculature at the stroke and below the stroke, acquired with a light sheet microscope 48 hrs following PT stroke of pre-clinical tests as described herein according to the invention;

FIG. 4e is a diagram illustrating stroke volume bar graph in saline- or NFPS-treated mice, calculated by an automated deep learning prediction model of pre-clinical tests as described herein according to the invention;

FIG. 4f is a diagram illustrating merged image demonstrating exact colocalization of AIVIA's automatic segmentation to raw data of pre-clinical tests as described herein according to the invention;

FIG. 4g is a diagram illustrating density of vessels in peri-infarct region in saline- or NFPS-treated mice of pre-clinical tests as described herein according to the invention;

FIGS. 4h and 4i are diagrams illustrating number of vessels in the stroke area according to diameter and to length. Data is mean±SEM; statistical significance p<0.05*, p<0.01**, p<0.001***, and p<0.0001**** of pre-clinical tests as described herein according to the invention;

FIGS. 5a to 5e are simplified block diagrams illustrating the process causing central nervous system cell death during an ischemic stroke;

FIG. 6 is a simplified block diagram illustrating a treatment using NMDAR antagonists to prevent cell death during an ischemic stroke;

FIGS. 7a to 7d are simplified block diagrams illustrating use of a glycine transporter-1 antagonist compound as a vascular dementia and/or stroke prophylactic agent according to a preferred embodiment of the invention; and,

FIG. 8 is a simplified block diagram illustrating an example compound of the class of glycine transporter-1 antagonist compounds for use as a vascular dementia and/or stroke prophylactic agent according to the preferred embodiment of the invention.

### DESCRIPTION OF THE PREFERRED EMBODIMENT

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described.

Throughout this application various publications are referenced by their full citations. The disclosures of these publications in their entireties are hereby incorporated by reference into this application in order to more fully describe the state of the art to which this invention pertains.

### Ischemic Stroke

Stroke is the leading cause of mortality and long-term disability. Further, direct and indirect costs such as hospital costs, rehabilitation, lack of productivity and poor quality of life persist for years after the ischemic event. Stroke is not only a sudden and devastating disease but also a subtle disorder. Evidence suggests that many people may have multiple “stroke events” during their life and the cumulative effects of these events can cause permanent physical and mental consequences.

Within the Central Nervous System (CNS), glycine serves as a neurotransmitter that facilitates excitatory neurotransmission at glutamartergic N-methyl-_{D}-aspartate (NMDA) receptors. The NMDA receptor is a ligand and voltage-gated ionotropic glutamate receptor that is widely expressed throughout the CNS. Activation of it relies on: binding of agonist-glutamate at the orthosteric binding site of the NMDA receptor GluN2 subunit with simultaneous binding of obligatory co-agonist at the GluN1 subunit strychnine-insensitive glycine-B binding site; and, concurrent membrane depolarization, which is required to expel a magnesium block from the channel pore. Stimulation of the NMDA receptor permits Ca²⁺ as well as K⁺ and Na⁺ influx, leading to neuronal excitation and intracellular signalling cascades.

### Pre-Clinical Research

The mechanisms of stroke and vascular dementia are similar, with the mechanism of vascular dementia resulting in a series of more frequently occurring “smaller strokes”. In vitro and in vivo pre-clinical research in the mechanism of strokes as described hereinbelow is disclosed in: “Cappelli, J., Khacho, P., Wang, B., Sokolovski, A., Bakkar, W., Raymond, S., Ahlskog, N., Pitney, J., Wu, J., Chudalayandi, P., Wong, A., & Bergeron, R. (2021). Glycine-induced NMDA receptor internalization provides neuroprotection and preserves vasculature following ischemic stroke. iScience, 25(1), 103539. https://doi.org/10.1016/j.isci.2021.103539”.

It is well established that mice represent a useful model for the simulation of human and other mammalian pathologies and assessment of potential therapeutic pathways and corresponding therapeutic efficacy. The research and results described herein utilizes such well established modeling.

### SUMMARY

Ischemic stroke is the second leading cause of death worldwide. Following an ischemic event, neuronal death is triggered by uncontrolled glutamate release leading to overactivation of glutamate sensitive N-methyl-_{D}-aspartate receptor (NMDAR). For gating, NMDARs require not only the binding of glutamate, but also of glycine or a glycine-like compound as a co-agonist. Low glycine doses enhance NMDAR function, whereas high doses trigger glycine-induced NMDAR internalization (GINI) in vitro. Following an ischemic event, in vivo, GINI also occurs and provides neuroprotection in the presence of a GlyT1 antagonist (GlyT1-A). Mice pretreated with a GlyT1-A, which increases synaptic glycine levels, exhibited smaller stroke volume, reduced cell death, and minimized behavioral deficits following stroke induction by either photothrom-bosis or endothelin-1. Moreover, in ischemic conditions, GlyT1-As preserve the vasculature in the peri-infarct area. Therefore, GlyT1 presents itself as a new target for the treatment of ischemic stroke.

### INTRODUCTION

Activity-dependent changes in N-methyl-D-aspartate receptor (NMDAR)-mediated synaptic strength are of great importance, because they serve as the molecular trigger for synaptic responses in many physiological and pathological processes such as ischemic stroke. Neuronal death following an ischemic event is triggered by uncontrolled glutamate release leading to the NMDAR overactivation on surrounding neurons, inducing excessive Ca²⁺-influx primarily through NMDARs (Wu and Tymianski, 2018). In physiological conditions, NMDARs require glutamate binding on the GluN2 subunit and glycine binding on the glycine binding site (GBS) on the GluN1 subunit (Rosenmund et al., 1998). Ascher's group showed that glycine (Johnson and Ascher, 1987), or a glycine-like substance (Kleckner and Dingledine, 1988), is a required co-agonist for NMDAR activation. Moreover, Salter and co-workers reported that high doses of glycine trigger GINI, in vitro, by promoting endocytosis of NMDAR through clathrin/dynamin-dependent machinery (Nong et al., 2003). Unlike constitutive internalization, which requires no channel activation (Nong et al., 2003; Nong et al., 2004), NMDAR internalization following glycine “priming” requires both glutamate and glycine present in the synaptic cleft.

Using a multidisciplinary and experimental approach, it has been found that during an oxygen-glucose deprivation paradigm (OGD), in vitro, not only glutamate but also an excess of glycine is released in the extracellular space. However, this is not sufficient to trigger GINI because the level of extracellular glycine is buffered by the glycine transporter type 1 (GlyT1) (Aragon et al., 1987; Guastella et al., 1992; Smith et al., 1992; Bergeron et al., 1998). Only when GlyT1s are antagonized, can glycine accumulate in the synaptic cleft and lead to robust NMDAR internalization. Photothrombosis (PT) and endothelin-1 (ET-1) are paradigms that mimic ischemic events, in vivo. It was observed from our results that both PT and ET-1 induced a significantly smaller stroke volume, less cell death and less behavioral deficits in mice in the presence of a GlyT1 antagonist (GlyT1-A), which increases glycine concentrations, and hence the occupancy of the GBS. Moreover, the neuroprotective effect induced by high occupancy of the GBS was further supported by preservation of the vascularization tree.

Overall, our experimental results indicate that increased levels of synaptic glycine before an ischemic event is a means of minimizing neuronal death. GlyT1-A administration before or shortly after an ischemic event, in vivo, triggers GINI and provides neuroprotection.

### Pre-Clinical Experimental Results

### High Concentrations of Glycine Induce NMDAR Internalization

First the effects of bath application of increasing glycine concentrations on stimulation-evoked NMDAR excitatory postsynaptic currents (NMDAR-EPSCs) recorded from CAI pyramidal neurons from acute hippocampal brain slices have been determined, as illustrated in FIG. 1a. At glycine concentrations below 250 μM, NMDAR-ESPC amplitudes were potentiated in a dose-dependent fashion (Johnson and Ascher, 1987; Bergeron et al., 1998; Forsythe et al., 1988; Paoletti et al., 1995). However, increasing the glycine concentration to 1 mM resulted in a significant decrease in NMDAR-EPSC amplitude (Nong et al., 2003; Han et al., 2013) and this effect was reversible, as illustrated in FIG. 1b. To verify that this decrease in amplitude was because of GINI, 1 mM glycine in the presence of 100 μM dynasore, a cell-permeable inhibitor of both dynamin-1 and dynamin-2, which blocks internalization (Nong et al., 2004; Kirchhausen et al., 2008) has been applied. As a result the decrease in NMDAR-EPSC amplitudes by 1 mM glycine was abolished in the presence of dynasore, as illustrated in FIG. 1c.

### Calcium Influx is Required for GINI to Occur

Previous studies have reported that the activity of dynamin is regulated by the Ca²⁺-sensitive phosphatase calcineurin (Lai et al., 1999; Traynelis et al., 2010). Therefore, the role of extracellular and intracellular Ca²⁺ on NMDAR-EPSC amplitudes in the presence of 1 mM glycine has been explored. The effects of various external Ca²⁺ concentrations on the NMDAR response to 1 mM glycine application have been examined. When 1 mM glycine was applied with low Ca²⁺ (1 mM), an increase in NMDAR-EPSC amplitude was observed, in contrast to what occurred with normal Ca²⁺ concentrations (3.5 mM). To further confirm the role of Ca²⁺ in GINI, a Ca²⁺ chelator BAPTA (10 mM) has been included, in the recording electrode. Here, a significant attenuation in the decrease in NMDAR-EPSC amplitude induced by 1 mM exogenous glycine has been observed. Moreover, extracellular application of 20 μM nimodipine, a L-type Ca²⁺ channel blocker, also attenuated GINI compared to control, further corroborating the data acquired with BAPTA. In contrast, depleting intracellular Ca²⁺ stores by incubating hippocampal slices for 1 hr in 30 μM cyclopiazonic acid (CPA), an inhibitor of intracellular Ca²⁺ pumps, had no effect on the glycine-induced decrease in NMDAR-EPSC amplitude, as illustrated in FIG. 1d. Together these data demonstrate that external Ca²⁺ influx across the plasma membrane is required for GINI to occur.

### Genetic Elevation of Extracellular Glycine Facilitates GINI

Heterozygous glycine transporter type 1 (GlyT1^{+/-}) mice exhibit a higher level of endogenous extracellular glycine (Gomeza et al., 2003; Tsai et al., 2004). Therefore, it was hypothesized that in these mice GINI could be triggered by lower doses of glycine. As illustrated in FIG. 1e, although there was no significant effect in the NMDAR-EPSC amplitude following bath application of 10 μM or 1 mM glycine between wild type (WT) and GlyT1^{+/}mice, bath application of 250 μM glycine, which potentiated the NMDAR-EPSC amplitude in WT mice, significantly inhibited the NMDAR-EPSC amplitude in GlyT1+/mice, as illustrated in FIG. 1f. The decrease of the NMDAR-EPSC amplitude induced by 250 μM glycine in GlyT1+/mice was abolished in the presence of dynasore, as illustrated in FIG. 1g. Therefore, the high levels of endogenous glycine in the GlyT1^{+/-} mice trigger GINI at lower exogenous glycine concentrations.

### Role of Glycine Binding Site Occupancy

In addition to glycine, D-serine also activates the GBS (Kleckner and Dingledine, 1988; Papouin et al., 2012). As illustrated in FIG. 1h, the effects of increasing D-serine concentrations on NMDAR-EPSC amplitudes in acute slices from WT mice was also dose-dependent. Moreover, the dose-response curve of NMDAR-EPSC amplitudes to D-serine was left-shifted relative to that of glycine because of its higher affinity to the GBS (Wolosker et al., 1999), as illustrated in FIG. 1i. The decrease in NMDAR-EPSC amplitude evoked following bath application of 1 mM D-serine was also abolished in the presence of dynasore, as illustrated in FIG. 1j.

Next it was investigated whether GINI could be modulated by increasing levels of either glycine or D-serine in WT mice. Glycine levels were increased via bath application of the selective GlyT1-A, N-[3-(4′-fluoro-phenyl)-3-(4′-phenylphenoxy) propyl] sarcosine (NFPS; 300 nM) (Aubrey and Vandenberg, 2001; Herdon et al., 2001; Mallorga et al., 2003; Liu et al., 2005; Pinto et al., 2015). As expected, there was a significant increase in evoked NMDAR-EPSC amplitude in the presence of NFPS alone (Bergeron et al., 1998). However, when NFPS was applied together with a potentiating concentration of D-serine (10 μM), a significant decrease in NMDAR-EPSC amplitude was observed, as illustrated in FIG. 1k. Interestingly, when NMDARs were first primed with high doses of glycine or D-serine, a subsequent application of a low dose of glycine or D-serine, also induced GINI.

Next, a transgenic mouse model was used in which the serine racemase gene was knocked out (SR^{-/-} mice) (Basu et al., 2009; Balu et al., 2012; Benneyworth and Coyle, 2012), as these mice exhibit low levels of _{D}-serine. In both WT and SR/mice, a low dose of D-serine (10 μM) potentiated the evoked NMDAR-EPSC amplitude. However, SR mice required a higher dose of D-serine (2 mM) than WT mice (1 mM) to induce a decrease in NMDAR-EPSC amplitude, as illustrated in FIGS. 1l and 1m. These findings demonstrate that there is a common mechanism of action for glycine or D-serine to trigger GINI. In addition, it has been found that GINI was neither sub-unit-specific nor attributed to AMPA receptor activity, and was not limited to the hippocampal region.

### Glycine is Released During Oxygen-Glucose Deprivation

Immunohistochemical data suggests that glycine may be co-localized in glutamatergic neurons (Cubelos et al., 2005); therefore, it has been hypothesized that depolarization of glutamatergic CAI pyramidal neurons during the oxygen-glucose deprivation (OGD) paradigm could result in detectable local glycine release (Rossi et al., 2007). To ensure glycine was released during an OGD paradigm, the sniffer-patch technique was used, wherein activation of glycine receptor ∞2 subunit indicated glycine release (Muller et al., 2013). When the OGD perfusate was applied to the slice, there was a marked increase in the frequency of channel opening in the patch and a significant increase in open probability (Pₒₚₑₙ) compared to control. Overall, these results strongly suggest that during OGD conditions, glycine is released into the CAI extracellular space. Given that multiple studies have demonstrated that glycine receptors (GlyRs) are only weakly expressed at CA1 hippocampal synapses (Muller et al., 2013; Hu et al., 2016; Chen et al., 2015), it has been speculated in the prior art that the target for the glycine release following OGD could be NMDARs.

OGD paradigm on acute slices, in vitro, decreases NMDAR current amplitude

In brief, it has been found that an OGD paradigm applied to acute slices during train stimulation induced NMDAR internalization. To further confirm that glycine is responsible, glycine oxi-dase (GO) was purified, an enzyme that catalyzes the breakdown of glycine. After demonstrating the effectiveness of purified GO on exogenous glycine levels, NMDAR-EPSC trains (20 Hz) were recorded with GO and the decrease of the NMDAR-EPSC amplitude was abolished following OGD. Altogether, these in vitro data demonstrate that glycine levels increase during ischemia; however, GINI is only triggered when glycine is further elevated using the train stimulation paradigm. Therefore, it was speculated that GINI could also be triggered in vivo during stroke in mice with elevated glycine levels.

Genetic elevation of brain glycine reduces infarct size following photothrombosis

Glycine has been shown to be neuroprotective in both in vitro (Hu et al., 2016) and in vivo models of stroke (Chen et al., 2015, 2017; Zhao et al., 2018; Qin et al., 2019); yet, proposed mechanisms have never been expanded into feasible pharmacotherapies. To determine if high glycine levels could result in a decrease in neuronal death following ischemia, a well-established focal ischemic paradigm, photothrombosis (PT) has been used. Because the in vitro data demonstrate that high glycine/D-serine levels are required to trigger GINI, one would expect that the stroke volume in GlyT1″ mice should be smaller than that observed in WT mice. Indeed, there was a statistically significant decrease in stroke volume in the GlyT1^{+/-} mice compared to WT. In contrast, stroke volumes were larger in SR mice, compared to WT, as illustrated in FIG. 2a.

Pharmacological elevation of brain glycine reduces infarct size following photothrombosis

To acutely increase the levels of endogenous glycine, WT mice were treated with NFPS 24 hrs pre-stroke (Aubrey and Vandenberg, 2001; Herdon et al., 2001; Mallorga et al., 2003; Liu et al., 2005). Forty-eight hours following PT stroke in both the saline- and NFPS-treated cohorts, stroke volume was quantified using 2,3,5-triphenyltetrazolium chloride (TTC) or via magnetic resonance imaging (MRI). The box-and-whisker plot shows a statistically significant decrease in median stroke volume in the NFPS-treated mice compared to the saline-treated mice, as illustrated in FIG. 2b. This decrease in infarct volume following NFPS treatment is consistent with what has been previously observed in the transient middle cerebral artery occlusion (tMCAO) model of ischemic stroke (Huang et al., 2016; Dojo Soeandy et al., 2019). In addition, FluoroJade C (FJC) staining demonstrated that the NFPS-treated mice also have significantly decreased levels of cell death compared with the saline-treated mice. Therefore, these data demonstrate that the blockade of GlyT1 is required for the reduction of stroke volume. Interestingly, this decrease in stroke volume was maintained when NFPS was administered up to 10 mins post-stroke, as illustrated in FIG. 2c.

Pharmacological elevation of brain glycine minimizes motor behavioral deficits following photothrombosis

Although encouraging, a decrease in stroke volume does not necessarily correlate with a decrease in post-stroke behavioral deficits (Pineiro et al., 2000). To determine if pre-treatment with NFPS could minimize post-stroke behavioral deficits, a well-established behavioral test of motor function, the adhesive removal test (Bouet et al., 2009) has been used. Following PT, a significant attenuation of post-stroke motor behavioral deficits was observed in the cohort of mice treated with NFPS in both time to contact and time to remove, with no significant stroke or drug effect on the unimpaired paw, as illustrated in FIG. 2c.

Pre-stroke administration of NFPS decreases stroke volume and improves motor behavioral deficits following endothelin-1 stroke

The PT stroke model does not recapitulate all of the clinical aspects of ischemia, particularly with respect to reperfusion of the infarct (Sommer, 2017). Therefore, to ensure that the observed decrease in stroke volume and attenuation of behavioral deficits was not an artifact of the PT stroke model, the experiments have been repeated using a second known model of focal stroke, the endothelin-1 (ET-1) model (Dojo Soeandy et al., 2019). Mice pre-treated with NFPS had significantly smaller ET-I stroke volumes compared with their sa-line-treated counterparts, as illustrated in FIG. 2e.

In the adhesive removal task, NFPS-treated mice showed significantly less post-stroke impairments in the impaired paw than the saline-treated mice, in both time to contact and time to remove, as illustrated in FIG. 2f. There was no significant stroke or drug effect on the unimpaired paw (Supplementary adhesive and cylinder task (Schallert et al., 2000) data for both stroke models and validation of ET-I model). The horizontal ladder test was an additional assessment of motor function (Metz and Whishaw, 2009). Following ET-1 stroke, there was a significant increase in impaired paw misses in the saline-treated group; however, in the NFPS-treated group, no significant increase in misses was observed. These data demonstrate that the blockade of GlyT1 ameliorated post-stroke out-comes in two models of stroke. Furthermore, this effect was not because of hypothermia. Therefore, these data emphasize the crucial role of GlyT1-A in the observed neuroprotection, and this is likely occurring because of GINI.

Blocking NMDAR internalization abolishes the neuroprotective effect of NFPS on stroke volume and behavior

GINI is driven by the recruitment of AP-2 and is mediated by A714 on the C-terminal domain of GluN1. Glycine priming for internalization is specific to A714; therefore, this residue is necessary for priming of NMDARs containing either GluN2A or GluN2B in recombinant systems (Han et al., 2013). To confirm that the in vivo observations are because of GINI, a point mutation was introduced into the NMDAR GluN1 subunit (A714L), which abolishes glycine-mediated NMDAR internalization in vitro (Han et al., 2013). First the functionality of the mutation was assessed via transient transfection of GluN1-WT or GluN1-A714L together with WT GluN2A subunit into HEK293 cells resulting in a functional NMDAR. Application of 1 mM glycine in cells expressing GluN1-WT induced a significant decrease in the amplitude of the NMDAR-EPSC, whereas in cells expressing GluN1-A714L this concentration significantly increased the NMDAR amplitude. To visually confirm that GINI was occurring, the movements of NMDARs were tracked over time by live-cell imaging following application of 1 mM glycine.

This GluN1 viral construct was then packaged into an adeno-associated virus (AAV) 2/9 and injected into the sensory-motor cortex of mice. The overall function of the NMDARs was reassessed in acute slices. A dose of 1 mM glycine did not decrease NMDAR-EPSC amplitudes in cells infected with the AAV-GluN1-A714L constructs. The spread of the virus occupied a volume that was comparable to the PT stroke, as illustrated in FIG. 3b, and there were no significant differences in the PT-induced stroke volume between the mice infected with either the AAV-GluN1-WT or the AAV-GluN1-A714L constructs, as illustrated in FIG. 3c. However, there was a significant decrease in stroke volume following pre-treatment with NFPS in mice infected with AAV-GluN1-WT. NFPS administration had no effect on stroke volume in the mice infected with AAV-GluN1-A714L.

The adhesive removal test was repeated on mice infected with either the AAV-GluN1-A714L mutation or the AAV-GluN1-WT. Administration of NFPS to the mice infected with AAV-GluN1-WT resulted in a significant decrease in post-stroke time to contact and time to remove in the impaired paw, as illustrated in FIG. 3d. Interestingly, in mice infected with the AAV-GluN1-A714L, as illustrated in FIG. 3e, there was no significant change in time to contact and time to remove following stroke in the NFPS-treated mice. The injections of the AAV-GluN1-WT or -GluN1-A714L alone had no effect on behavior. Taken together, these data confirm that GlyT1-A administration induces neuroprotection in vivo, via GINI.

### Pre-Stroke Administration of NFPS Attenuates Vascular Dysfunction

Stroke is primarily characterized as a vascular disease; therefore, the impact of NFPS on vascular function and morphology following PT stroke has been evaluated. Using Laser Doppler flowmetry (LDF), it was found that PT stroke induced a significant decrease in blood flow and this effect was rescued with NFPS pre-treatment, as illustrated in FIG. 4a. In GlyT1^{+/-} mice, there was no significant change in blood flow following stroke. Interestingly, in SR mice, PT stroke induced a highly significant decrease in blood flow, as illustrated in FIG. 4b.

Next it was assessed if NFPS could modify vascular morphology by pairing transcardial perfusions of a fluorescent dye with tissue clearing and light sheet fluorescence microscopy (LSFM). This strategy allowed for complete labeling of the cerebral vasculature, as illustrated in FIG. 4c. First a deep learning segmentation model was used to automatically calculate stroke volume from the cleared tissue, as illustrated in FIGS. 4d and 4e. A decrease in stroke volume in NFPS-treated mice compared to saline-treated mice was observed, as illustrated in FIG. 4e. These results are consistent with data illustrated in FIG. 2b. Further the effect of NFPS following PT on vascular density was explored. The PT-induced decrease in vascular density was attenuated with NFPS treatment compared to saline-treated mice, in the peri-infarct region, as illustrated in FIGS. 4f and 4g. Furthermore, NFPS pre-treatment decreased the PT-induced loss in vessels of smaller diameter and length, as illustrated in FIGS. 4h and 4i, compared to saline-treated mice, in the peri-infarct region. Taken together, treatment with NFPS before an ischemic event protects the function and morphology of the cerebral vasculature.

### Discussion

The results demonstrate that during an ischemic event, not only glutamate but also glycine is released in the extracellular space. In such ischemic conditions, when GlyT1s are antagonized, glycine accumulates in the synaptic cleft, reaches the “set point,” and triggers GINI. This is the first report demonstrating that GINI occurs in vivo, provides neuroprotection, and preserves brain vasculature.

Using whole-cell patch-clamp recordings, dose-response curves were generated and the effects of glycine on NMDAR current amplitudes were measured. It has been observed that application of low concentrations of glycine (≤250 μM) increased the NMDAR-EPSC amplitudes (Johnson] and Ascher, 1987, 1992). Paradoxically, it was found that application of high concentrations of glycine (>1 mM) significantly reduced the NMDAR-EPSC amplitudes. This internalization of NMDARs has been reported to be triggered by an increase in NMDAR binding to intracellular clathrin/dynamin-dependent endocytic machinery (Nong et al., 2003; Han et al., 2013). Because the role of GlyT1s is to keep glycine concentrations below the saturating level of the GBS on NMDARs (Furukawa and Gouaux, 2003) the relevance of the pivotal work from Salter and co-workers was questioned by several groups. This low synaptic concentration of endogenous glycine is far from the concentration required to trigger GINI (Aragon et al., 1987; Guastella et al., 1992; Smith et al., 1992; Bergeron et al., 1998). As the effect of different doses of glycine on NMDAR-EPSCs appears to match that of the “inverted-U” shaped curve, the relationship between glycine levels and NMDAR internalization was investigated using in vitro ischemic paradigms. There was evidence that synaptic NMDARs internalize following elevation of glycine during a train of stimuli during OGD, an in vitro model of ischemia (Rossi et al., 2000). Moreover, the application of a high concentration of glycine or D-serine not only triggers GINI but also primes NMDARs for GINI. When a high dose is applied and washed off before application of a low dose of one of the co-agonists, GINI is induced.

Interestingly, it has been demonstrated, in vivo, that elevation of extracellular glycine by pharmacological blockade or genetic deletion of GlyT1 resulted in a decreased stroke volume and an attenuation of motor deficits in mice following ischemic stroke induced by PT or ET-1. This was observed when NFPS was administered 24 h pre-stroke, or up to 10 min post-stroke. There is also evidence that GINI, in vivo, is directly modulating the GluN1 subunit of NMDAR channel function during ischemic stroke as the effect of NFPS on both stroke volume and behavior is completely abolished when mice are focally infected with a viral vector expressing a non-internalizing GluN1 receptor subunit (AAV-GluN1-A714L) (Han et al., 2013).

The NMDAR co-agonist, glycine, has been previously shown to be neuroprotective in both in vitro (Hu et al., 2016) and in vivo models of stroke (Chen et al., 2015, 2017, 2020; Zhao et al., 2018; Qin et al., 2019). However, the mechanism by which glycine affords neuroprotection during stroke in vivo remains elusive. Recent work suggests that it is via modulation of intracellular pathways, including the Phosphatase and tensin homolog (PTEN)/protein kinase B (AKT) signaling pathway (Qin et al., 2019; Zhao et al., 2018), or vascular endothelial growth] factor receptor 2 (Chen et al., 2020). Glycine is also thought to exert its neuroprotective effects via mediation of non-ionotropic NMDAR function (Chen et al., 2015, 2017; Hu et al., 2016), or by promoting microglial polarization (Liu et al., 2019). Partial agonists at the GBS on NMDARs also afford neuro-protection following an OGD challenge (Stanton et al., 2009) and during MCAO paradigm (Zheng et al., 2017). Pharmacological elevation of brain glycine following NFPS administration potentiates ischemic pre-conditioning (Pinto et al., 2015) and confers neuroprotection via global activation of ionotropic GlyRs during transient MCAO (Huang et al., 2016). Overall, there are many ways in which glycine has been shown to be neuroprotective, all of which may be occurring in conjunction with GINI. However, here it is determined for the first time the important role of GlyTs as the blockade of these transporters minimized cell death following an ischemic stroke in an in vivo model.

It has been suggested that glycine and D-serine may be therapeutically beneficial by down regulating NMDARs, such as rodent models of traumatic brain injury and lipopolysaccharide-induced neuroinflammation (Biegon et al., 2018). There is also a growing body of evidence to suggest that extracellular glycine is neuroprotective in several rodent ischemic stroke models (Huang et al., 2016; Zheng et al., 2017; Zhao et al., 2018; Liu et al., 2019; Chen et al., 2020; Yamamoto et al., 2016). Moreover, the level of extracellular glycine appears to be important in stroke outcome. A low level of glycine, corresponding to increased NMDAR activation, appears to be deleterious. In contrast, an elevated level of glycine appears to be neuroprotective (Yao et al., 2012). These findings are in agreement with our data and results described herein. Indeed, the transgenic GlyT1^{+/-} mice, which have high endogenous levels of glycine and consequently a high occupancy of the GBS, are more resistant to PT, whereas the SR^{-/-} mice, which have a low occupancy of the GBS, are more sensitive to PT challenge.

Despite an overwhelming body of evidence from animal studies that implicate NMDARs in neuronal loss (Gotti et al., 1988; Park et al., 1989; Scatton, 1994; Prass and Dirnagl, 1998), all clinical trials of drugs targeting one of the numerous binding sites on NMDARs have failed because of poor tolerance or lack of efficacy (Ikonomidou and Turski, 2002; Lipton, 2004; Kalia et al., 2008). One reason for this may be the difficulty in obtaining a therapeutic degree of NMDAR-blockade that does not interfere with critical NMDAR-dependent functions in neuronal circuits (Kostandy, 2012). The widespread inhibition of NMDAR function is not compatible with baseline synaptic transmission. As such, our data and the results described herein demonstrate that attention should turn to modulation of NMDAR function during stroke. In this study, it has been shown that GINI is not a direct antagonism of NMDARs but rather a dynamic and reversible phenomenon which dampens NMDAR-mediated excitotoxicity during ischemia while maintaining basal synaptic activity of NMDARs.

The complex vascular network of the brain and its integrity are essential for normal brain function. Following an ischemic event, the delivery of oxygen and nutrients to neurons and glial cells are impaired. Because the brain is highly vulnerable to compromises in blood supply, the potential impact of NFPS in preserving brain vasculature was investigated. It has been previously reported that changes in microvasculature, such as density and diameter, correlate with disease states (Bennett et al., 2017). PT stroke induced a decrease in vascular density in the peri-infarct region. Our data and the results described herein demonstrate that this decrease was attenuated by a pre-treatment with NFPS. Histogram analysis shows that vessels of 2-3 μm in diameter were the most affected post-stroke. Application of NFPS decreased the size of vessels occluded, suggesting that the peri-infarct region could undergo enhanced vascular remodeling during the recovery period. It cannot be concluded that the mechanism underlying this observation is directly linked to GINI. However, increasing the level of endogenous glycine with NFPS protects the vascular network following stroke, and ultimately leads to improved behavior outcomes.

Overall, our data and the results described herein demonstrate that elevation of glycine via blockade of GlyT1s before or shortly after an ischemic event provide a rationale for the repurposing of currently approved pharmaceuticals with a similar mechanism of action as potential stroke treatments. For example, the glycine reuptake blocker sarcosine is authorized for clinical use in the treatment of schizophrenia at daily doses of 1-2 g per day and is well tolerated in these patients (Gibert-Rahola and Villena-Rodriguez, 2014; Strzelecki et al., 2014, 2015; Amiaz et al., 2015; Lin et al., 2017). Because the chronic administration of GlyT1-As have been proven to be safe, and we observe the most robust neuroprotective effect when GlyT1-As are administered prestroke, it is expected GlyT1-As to potentially be utilized as a preventative strategy for stroke, particularly considering that the in vivo data demonstrate the pre-clinical efficacy of this class of drugs in minimizing the deficits induced by PT and ET-1 paradigms, and considering that several GlyT1-As have been tested and proven to be safe and well tolerated in human clinical trials (Harvey and Yee, 2013; Shahsavar et al., 2021). Accordingly, GlyT1 should be clinically tested for a new therapeutic for ischemic stroke.

### EXPERIMENTAL MODEL AND SUBJECT DETAILS

### Animals

All procedures in the research described herein were carried out on female and/or male 8-10-week-old mice in accordance with the guidelines of the Canadian Council on Animal Care and approved by the University of Ottawa Animal Care Committee. The following transgenic mouse lines were utilized: heterozygous glycine transporter type 1 (GlyT1^{+/-}), serine racemase knockout (SR−/−), and N-methyl-_{D}-aspartate receptor (NMDAR) GluN2A subunit knockout (GluN2A−/−) mice (Kannangara et al., 2015), along with their wild type (WT) litter mates (on C57Bl/6;S129, C57BI/6;S129 and C57Bl/6 backgrounds respectively). In vivo behavioural experiments were performed on C57BI/6 WT mice from Charles River®. The animals were housed under standard conditions and had access to chow and water ad libitum.

### Cell Lines

Cell culture, imaging, and electrophysiological experiments were carried out on Human Embryonic Kidney (HEK293) cells. Sniffer-patch experiments were performed on Chinese Hamster Ovary (CHO) cells.

### Method Details

### Electrophysiology

Whole-cell electrophysiology on hippocampal slices and HEK293 cells. Whole-cell voltage-clamp recordings were obtained from visually identified CA1 pyramidal cells from acute hippocampal brain slices (300 μm thick) in oxygenated artificial cerebrospinal fluid (ACSF) as previously described (Martina et al., 2004, 2005). The cells were voltage-clamped at −65 mV using cesium methane sulfonate based internal solution and postsynaptic currents were evoked by electrical stimulation of the Schaffer collaterals with a bipolar stimulating electrode positioned in the Stratum radiatum. The intensity of the stimulation was adjusted to obtain evoked excitatory postsynaptic currents (EPSCs). The stimulation protocol consisted of a single 100 us current pulse (10-200 μA) evoked every 12s. For the train stimulation protocol, 10 current pulses (100 us long) were evoked at 50 Hz for 200 ms and then repeated once every 20s. To isolate the NMDAR-EPSC, a low concentration of MgCl₂ (0.13 mM) ACSF was used wherein the CaCl₂ concentration was increased to 3.5 mM to maintain cation balance.

HEK293 cells were used for electrophysiology recordings 48-72 hrs following transfection with either the pHluorin-GluN1-WT or pHluorin-GluN1-A714L cDNA along with an equimolar ratio of the GluN2A. NMDAR currents were evoked using pressure ejection (10 psi) from a picospritzer micropipette filled with 10 μM glycine and 1 mM glutamate (Sigma-Aldrich) for a duration of 25-50 ms every 20s at a membrane potential of −60 mV. HEK293 cells were recorded in HEPES-buffered saline external solution with low MgCl₂ using a potassium gluconate recording solution.

When required, additional drugs were applied including various concentrations of D-serine and glycine (Millipore Sigma), as well as 300 nM N-[3-(4′-fluorophenyl)-3-(4′-phenylphenoxy) propyl] sarcosine (NFPS; Tocris Bioscience), 10 mM BAPTA (Thermo Fisher), 30 μM cyclopiazonic acid (CPA) (Tocris Bioscience), and 20 μM nimodipine (Milli-pore Sigma). The inhibitors of clathrin-mediated endocytosis, 100 μM dynasore (Millipore Sigma), 100 μM dynamin blocking peptide (DBP; Tocris Bioscience), and 30 μM Dyngo4a (Abcam) were included in the internal solution.

### Sniffer-Patch Technique and OGD Paradigm

Sniffer-patch technique. To detect glycine release, the “sniffer patch” technique (Allen, 1997; Lee et al., 2007a; Aubrey et al., 2007; Scain et al., 2010) was used. A Chinese Hamster Ovary (CHO) cell line stably transfected with the 1 alpha 2 subunit of glycine receptor (GlyR) was generated (Mangin et al., 2003). Outside-out membrane patches were excised from the CHO cells using thick-walled borosilicate glass pipettes filled with a cesium chloride internal solution.

Following patch excision, the electrode was placed in the Stratum radiatum of the CA1 region of the hippocampus to detect glycine release and allow channel activation. Channel open probability (Pₒₚₑₙ) was derived by measuring the mean open time of all the single channel events during the recording window, then dividing by the sum of the mean open and shut times. Multiple channel openings were set as a Pₒₚₑₙ=1 for that particular time period.

Oxygen-glucose deprivation paradigm. To mimic ischemia, the acute slices were challenged by an oxygen-glucose deprivation paradigm (OGD) modified from Rossi et al. (Rossi et al., 2000). In this paradigm, external glucose was replaced with 7 mM sucrose, and the external solution was saturated with 95% N₂/5% CO₂ instead of 95% O₂/5% CO₂. Iodoacetate and cyanide were also added to the OGD external solution to block glycolysis and oxidative phosphorylation.

Purification of glycine oxidase. The plasmid containing His-tagged glycine oxidase (GO) was generated from Bacillus subtilis. This plasmid was a gift from Dr. Steven Ealick (Cornell University, NY, USA). The protein was expressed in E. coli and purified as previously described (Job et al., 2002; Settembre et al., 2003; Molla et al., 2003; Pedotti et al., 2009; Caldinelli et al., 2009).

### Surgical Procedures

Photothrombosis and endothelin-1 stroke. NFPS or a vehicle control solution was injected intraperitoneally (i.p.) into C57BI/6 mice either 24 hrs prior to stroke or 10 mins/60 mins/120 mins post-stroke, at a dose of 5 mg/kg. Photothrombotic (PT) (Lee et al., 2007b) or cortical endothelin-1 (ET-1) (Wang et al., 2007) strokes were induced as previously described. Mice were anesthetized with 2.5% isoflurane in O₂ and mounted onto a stereotaxic frame. For PT strokes, a dose of 10 mg/mL of Rose Bengal (Tocris) was injected i.p. Immediately following the injection of the dye, the skull was exposed to visualize bregma. Using the stereotaxic device, a 520 nm laser (˜20 mW; Beta Electronics) was positioned above the sensorimotor cortex (AP+0.7, ML +2.0) and turned on for 10 mins to induce a permanent occlusion. For ET-1 strokes, once the skull was exposed and a craniotomy performed for each injection site (1. AP +0.0, ML +2.0, DV −1.6; 2. AP +0.2, ML +2.0, DV −1.4; 3. AP +0.4, ML +2.0, DV −1.3), 1 μL of 2 μg/μL human, porcine ET-1 (Abcam), dissolved in 2.7 μg/μL L-NAME (Abcam), was injected over 5 mins with a 28G 10 μL Hamilton syringe to induce a transient ischemic stroke.

Cortical infection with AAVs. Mice were anesthetized with 2.5% isoflurane in O₂ and mounted onto a stereotaxic apparatus. The intact skull was exposed to visualize bregma. The following injection sites were measured from bregma: 1. AP +1.2, ML +2.0, DV −0.5; 2. AP +0.2, ML +2.0, DV −0.5. A craniotomy was performed at each site prior to injecting 0.5 μL of 10-12 PFU/mL (plaque forming units) of either a AAV-WT-GluN1 or the mutant AAV-GluN1-A714L construct, over 5 mins with a 28G 10 μL Hamilton syringe. Please refer to section entitled “Generation of WT and A714L viral constructs”, below, for more information.

Laser Doppler Flowmetry. Laser Doppler Flowmetry (LDF) recordings following PT were performed as previously described (Toussay et al., 2019). Mice were anesthetized with an i.p. injection of a 0.01 ml/g cocktail consisting of 120 mg/kg ketamine and 10 mg/kg xylazine, and then mounted onto a stereotaxic apparatus. Following exposure and thinning of the skull, the laser probe (Transonic Systems) was positioned over the sensory motor cortex (AP +0.7, ML +2.0) and baseline activity was recorded for 5 mins. The laser probe was replaced with a 520 nm laser (˜20 mW; Beta Electronics) to induce PT stroke, as described above. Following PT, LDF recordings were performed for an additional 30 mins.

### A714L Generation, Imaging, and In Vivo Spread Quantification

Generation of WT and A714L viral constructs. The GluN1 constructs were made by cloning the GluN1 coding region of a SuperEcliptic Phluorin (SEP)-tagged GluN1 construct (Addgene #23999) (Choi et al., 2014) into pDrive cloning vector (pDrive cloning vector, Qiagen). This was then used as a template to create the A714L mutant clone by site-directed mutagenesis. These cDNAs were used for transfection of HEK293 cells. For generation of GluN1-WT and GluN1-A714L adeno-associated virus (AAV), the coding fragments of these constructs were sub-cloned into an adeno-associated viral vector, and viral constructs were then packaged with plasmid AAV2/9 at the University of Laval.

NMDAR internalization imaging in HEK293 cells. HEK293 cells were transiently transfected with either pHluorin-GluN1-WT or pHluorin-GluN1-A714L cDNAs together with GluN2A cDNA. HEK293 cells were then grown for 24-48 hrs in the presence of D-APV (Tocris Bioscience). Images were acquired with an LSM880 Confocal Microscope (Zeiss), with cells in a modified HEPES buffer (add 1 mM Glutamate, omit 0.13 mM MgCl₂). A 647 nm-tagged FluoTag®-X4 anti-GFP (1:250-1:500; NanoTag Biotechnologies) was added to tag extracellular NMDARs prior to the acute application of an internalizing dose of glycine. The nanotags (anti-GFP nanobody) are cell impermeable and tag NMDARs on the cell surface. Therefore, the nanotags were observed within the cell only when NMDARS had been internalized. Images were acquired every 3 mins over 10-12 mins to visualize internalization. Internalization was deemed to have occurred when the cell-impermeable NanoTag (647) was observed within the cell.

Viral spread quantification. Three weeks following cortical infection, mice were transcardially perfused with 1×PBS, followed by 4% PFA. Brains were sliced into 0.5 mm coronal slices and cleared following the SeeDB tissue clearing protocol described by Ke et al. (Ke et al., 2013). Following tissue clearing, brains were imaged with the Zeiss LSM800 confocal microscope. All viral spread analysis was performed manually using Fiji (ImageJ.com).

### Immunofluorescence, Stroke Volume Quantification and Cell Death Assay

Immunohistochemistry. Antigen retrieval was performed on slices before being washed then permeabilized with 0.25% (w/v) gelatin and 0.2% Triton X-100 (v/v) in PBS (PBS-GT). Slices were incubated in primary antibodies in PBS-GT, then washed three times with PBS-GT before the addition of fluorescent secondary antibodies at room temperature (RT). Slices were rinsed, air-dried, and mounted onto slides (Table S1) (Muller et al., 2013). Platelet endothelial cell adhesion molecule-1 (CD-31:647 nm) and Collagen IV (CollIV; 647 nm) were depicted in purple and visually colocalized to filled, perfused vessels (488 nm).

Quantification of stroke volume-magnetic resonance imaging. Magnetic resonance imaging (MRI) was performed at the University of Ottawa pre-clinical imaging core using a 7 Tesla GE/Agilent MR 901. Mice were anaesthetized for the MRI procedure using isoflurane in O₂: induction at 3%, maintenance at 1.5%. A 2D fast spin echo sequence (FSE) pulse sequence was used for the imaging, with the following parameters: slice thickness=0.5 mm, spacing=0 mm, field of view=2.5 cm, matrix=256×256, echo time=41 ms, repetition time=7000 ms, echo train length=8, bandwidth=16 kHz, fat saturation. Stroke lesions demonstrated hyperintensity.

Quantification of stroke volume-triphenyltetrazolium chloride. Stroke volume quantification was performed using 2,3,5-triphenyltetrazolium chloride (TTC; Sigma) (Hatfield et al., 1991; Benedek et al., 2006). Forty-eight hours post-stroke, mice were deeply anesthetized with 5% isoflurane in O₂ before decapitation for slicing on a vibratome (Leica) in cold ACSF at 0.5 mm. Slices were incubated in 2% TTC at 37° C. for 10 mins, then transferred to 4% paraformaldehyde (PFA) at 4° C. Brain slices were imaged from both sides and the surface area of the infarct regions were measured on Fiji (ImageJ.com) and multiplied by the thickness of the slice to obtain a final volume.

Quantification of stroke volume-cresyl violet. Forty-eight hours post-stroke, mice were transcardially perfused with 1×PBS, followed by 4% PFA. Brains were collected and post-fixed in 4% PFA overnight and then incubated in sucrose until saturation. Serial 25 μm thick coronal sections were cut on a cryostat (Microm HM500), and collected onto positively charged Superfrost Plus Microscope Slides (Fisher Scientific). The slides were immersed in xylene and then rehydrated in decreasing concentrations of ethanol before being placed in double distilled water (ddH₂O). Once rehydrated, slides were stained with cresyl violet (CV) (Electron Microscopy Sciences) and placed in ddH₂O. Slides were then dehydrated in increasing concentrations of ethanol before being immersed in xylene. Once removed, the slides were mounted with DPX mounting media (Sigma). Images of CV-stained slices were acquired with the EVOS FLAuto2 inverted epifluorescence microscope under brightfield. The surface area of the infarct regions was multiplied by the distance between each collected slice (500 μm) to obtain a volume. The sum of all slices was used to obtain a final stroke volume per brain.

Quantification of neuronal loss-FluoroJade C. The brain tissue preparation for FluoroJade C (FJC; EMD Millipore) was treated exactly as that of the CV brain tissue. Slides were first immersed in 1% sodium hydroxide in 80% ethanol, 70% ethanol and finally in ddH₂O before being incubated in a 0.06% potassium permanganate (Sigma-Aldrich) solution. This was followed by an incubation in a 0.0001% FJC solution dissolved in 0.1% aqueous acetic acid and combined with 0.0001% DAPI (Santa Cruz Biotechnology), and slides were once again rinsed with ddH₂O and left to air dry. Slides were then immersed in xylene and mounted with FluoroMountG (Sigma-Aldrich) (Ehara and Ueda, 2009). Imaging was completed with the Zeiss AxioObserver Z1 inverted epifluorescence microscope using GFP (488/509 nm) and DAPI (359/461 nm) filters. Analysis of the total number of degenerating neurons was performed using IMARIS 9.2 (Bit-plane). IMARIS 9.2 was set to detect and count all green (representing degenerating neurons) and blue (representing nuclear DNA) spots on each image and then calculate colocalization. The cells having been tagged by both DAPI and FJC were counted as FJC positive neurons.

### Tissue Clearing Light Sheet Microscopy

Tissue clearing. CUBIC tissue clearing was completed as previously described (Matsumoto et al., 2019). Following perfusion, the tissue was post-fixed overnight in 4% PFA, then washed in 1×PBS the following day. Following washes, tissue was submerged in half diluted CUBIC-L (1:1, CUBIC-L: Water) at 37° C. over-night. Tissue was submerged in CUBIC-L at 37° C. with gentle shaking over 10 days, changing the solution every 48 hrs. Tissue was then washed in IX PBS before being submerged in half diluted CUBIC-R⁺(M) (1:1, CUBIC-R⁺(M): Water) overnight at RT with gentle shaking. Tissue was submerged in CUBIC-R⁺(M) the following day, then replaced with fresh CUBIC-R⁺(M) 24 hrs later. Tissue was imaged with a light sheet microscope in a refractive index matched imaging solution consisting of a mixture of HIVAC-4 with mineral oil.

Imaging and segmentation. Imaging was performed using our custom-built light sheet microscope. CUBIC-cleared brains were imaged using a 2.5× objective (NA0.07), 488 nm excitation laser line and 5 μm steps. Each sample was scanned as a series of tiles, then stitched into a single image using TeraStitcher. The stitched scans were then run through AIVIA 9 to segment and create 3D reconstructions of the vascular network. Properties of each vessel (diameter and length) were automatically calculated by Al-VIA 9, and then exported for analysis.

Automated quantification of stroke volume (stroke volume prediction). Stroke volume in cleared tissue was calculated as follows: the areas representing stroke in each slice were identified, multiplied by their z-depth (thickness), then summed to obtain a total volume. The stroke regions were identified in each slice using a deep convolutional neural network (Kermany et al., 2018; Biswas and Barma, 2020; Yu et al., 2018) which was deemed 98% accurate. The network was first pre-trained on a large data-set (Deng et al., 2009), then further trained using 906 experimental scans.

Post-stroke vascular morphology quantification. For analysis of vascular morphology in the peri-infarct region, transcardial FITC-BSA staining was paired with CUBIC brain clearing to allow for LSFM imaging. Forty-eight hours post PT stroke, mice were transcardially perfused with 20 mL 1×PBS, then 20 mL 4% PFA. Mice were then submerged in a 37° C. water bath, facing down at an angle of 30° before being perfused with 10 mL of 0.5% FITC-BSA (Sigma-Aldrich), in 2% gelatin (Sigma-Aldrich). Subsequently, mice were submerged in an ice bath for 30 mins before the brain was dissected out (Tsai et al., 2009). Brains were collected and post-fixed in 4% PFA overnight, then cleared following the CUBIC tissue clearing protocol. Following tissue clearing, brains were imaged by LSFM. A 1.125 mm³ region of interest lateral to the stroke site was manually selected, then analyzed using AIVIA 9 (DRVISION Technologies).

### Behavioural Tests

Adhesive removal test. The adhesive removal test was performed as described (Bouet et al., 2009). Mice were trained pre-stroke daily over 5 days and tested post-stroke over 2 days. Trials began with 1 min of habituation to an empty home cage, before strips of adhesive were placed onto both forepaws. The mouse was then placed back into the cage and the times to contact and remove the adhesive strips were recorded by two experimenters. Mice were allotted a maximum of 2 mins to complete the task. The times to contact and remove the pieces of adhesive tapes were compared per paw as well as pre- and post-stroke.

Horizontal ladder test. The horizontal ladder test was performed based on protocols described previously (Farr et al., 2006; Metz and Whishaw, 2009) with slight modifications. Mice underwent one day of training prior to stroke. In the pre-stroke trials, mice crossed the ladder up to seven times or until they had performed two acceptable runs. In turn, during the post-stroke trials, mice had three attempts to cross the ladder, two of which were scored. Each trial was recorded with a video camera. Scoring and analysis were performed by an experimenter blind to the conditions. The video recordings of the best two trials from each mouse were analyzed frame-by-frame with Noldus Observer XT program. Each limb's step was scored as either “correct”, “partial” or “miss”. The percentage of missed steps pre- and post-stroke were compared.

Cylinder test. The cylinder test was performed based on protocols described (Schallert et al., 2000; Bal-kaya et al., 2013), with slight modifications. Mice were placed in a transparent cylinder and filmed with an overhead camera until they reared 22 times. With each rear, three types of behaviours were recorded: (A) right paw is exclusively weight bearing; (B) left paw is exclusively weight bearing; (C) both paws are weight bearing at the same time. The video recordings of each trial were analyzed frame-by-frame with Noldus Observer XT program by a single experimenter blind to the conditions. The length and frequency of forelimb contacts to the wall of the cylinder were scored. The behaviours were expressed per paw as an average time in relation to the sum of the independent left and right weight bearing. The average time spent on the impaired paw (right) was compared in pre-stroke and post-stroke trials.

### Quantification and Statistical Analysis

### Quantification and Statistical Analysis

All data are presented as means±S.E.M.; n represents the number of mice or cells in each group, as indicated in each figure. In most cases, statistical significance was determined by a paired, two-tailed Student's t-test or a two-way repeated measure ANOVA followed by Bonferroni post hoc comparisons, for multiple group comparisons. In cases in which datasets had multiple missing values, a mixed-model (ANOVA) was implemented by GraphPad Prism. For analyses of groups across multiple time points, a one-way ANOVA was utilized. Statistical analyses and data presentation were completed using both OriginPro 8.5 (Origin-Lab Software) and GraphPad Prism 8 (GraphPad Software).

For electrophysiological data, decay kinetic and amplitude analysis were performed on averaged traces. Decay time constants were best fit with a double exponential function and expressed as a weighted mean. Due to summation, EPSC amplitudes in the train were measured from the end of the previous EPSC rather than from the initial baseline.

### LIST OF ABBREVIATIONS

AAV Adeno associated virus;
ACSF Artificial cerebrospinal fluid;
CHO Chinese Hamster Ovary;
CPA Cyclopiazonic acid;
CV Cresyl violet;
DBP Dynamin blocking peptide;
ddH2O Double distilled water;
EPSCs Excitatory postsynaptic currents;
ET-1 Endothelin-1;
FJC FluoroJade C;
GBS Glycine binding site;
GINI Glycine-induced NMDAR internalization;
GluN2A^{-/-} NMDAR GluN2A subunit knockout;
GlyR Glycine receptor;
GlyT1 Glycine transporter type 1;
GlyT1^{+/-} Heterozygous glycine transporter type 1;
GlyT1-A Glycine transporter type 1 antagonist;
GO Glycine oxidase;
i.p. Intraperitoneal;
LDF Laser Doppler flowmetry;
LSFM Light sheet fluorescence microscopy;
tMCAO Transient middle cerebral artery occlusion;
MRI Magnetic resonance imaging;
NFPS N-[3-(4′-fluorophenyl)-3-(4′-phenylphenoxy) propyl] sarcosine;
NMDAR N-methyl-_{D}-aspartate receptor:
NMDG N-methyl-_{D}-glucamine;
OGD Oxygen-glucose deprivation paradigm;
PBSG 0.25% (w/v) gelatin in PBS;
PBSGT 0.25% (w/v) gelatin and 0.2% Triton X-100 (v/v) in PBS;
PFA Paraformaldehyde;
Pₒₚₑₙ Open probability;
PT Photothrombosis;
RT Room temperature;
SR−/− Serine racemase knockout;
TTC 2,3,5-triphenyltetrazolium chloride;
VGAT Vesicular GABA transporter;
VGlut Vesicular glutamate transporter;
WT Wild type.

FIGS. 5a to 5e schematically illustrate the process causing CNS cell death during an ischemic stroke. During an ischemic stroke a blood clot blocks the provision of oxygen (O₂) and glucose to cells of the CNS. The cells become energy deprived and dysregulated, as illustrated in FIG. 5a. Consequently, the cells release a large amount of Glycine (Gly) and Glutamate (Glu), causing an NMDAR over-activation, as illustrated in FIG. 5b. Gly and Glu then bind to the NMDAR and open it, i.e. provide an open access channel to the inside of the cell, as illustrated in FIG. 5c. The open access channels enable a large influx of Calcium ions (Ca²⁺) into the cell, causing cell death, as illustrated in FIGS. 5d and 5e.

Directly blocking the NMDAR receptors using NMDAR receptor antagonists substantially reduces the influx of Ca²⁺ into the cell, resulting in less NMDAR mediated excitotoxicity during ischemic stroke which is neuroprotective and substantially prevents cell death, as illustrated in FIG. 6. However, it has been found that directly blocking the NMDARs is not a viable treatment option. It did not provide any improvement and caused psychomimetic symptoms in many patients.

### Administration of a Glycine Transporter-1 Antagonist Compound

Referring to 7a to 7d, a use of a compound of the class of glycine transporter-1 antagonist compounds as a vascular dementia and/or stroke prophylactic/treatment agent according to the invention is provided. An example compound of the class of glycine transporter-1 antagonist compounds for use as a vascular dementia and/or stroke prophylactic/treatment agent is illustrated in FIG. 8.

Administration of a compound of the class of glycine transporter-1 antagonist compounds results in a blockage of Glycine-transporter-l's and consequently in a increase of glycine levels between the cells, which primes the NMDARs for internalization into the cell, as illustrated in FIG. 7a. During an ischemic stroke, glutamate and glycine are released. The released glutamate and glycine bind to the NMDARs and cause some NMDARs to open and letting Ca²⁺ into the cell while causing most NMDARs to move into the cell and, therefore, are unable to let Ca²⁺ into the cell, as illustrated in FIGS. 7b and 7c. The substantially reduced number of open NMDARs at the cell surface substantially reduces the influx of Ca²⁺ into the cell, resulting in substantially reduced cell death during ischemic stroke conditions, as illustrated in FIG. 7d.

The glycine transporter-1 antagonist compounds for use as a vascular dementia and/or stroke prophylactic/treatment agent according to the invention can be administered orally in the form of tablets or capsules, thus enabling easy self-administration thereof to achieve a therapeutically effective dosage.

### LIST OF REFERENCES AS SET OUT IN

“Cappelli, J., Khacho, P., Wang, B., Sokolovski, A., Bakkar, W., Raymond, S., Ahlskog, N., Pitney, J., Wu, J., Chudalayandi, P., Wong, A., & Bergeron, R. (2021). Glycine-induced NMDA receptor internalization provides neuroprotection and preserves vasculature following ischemic stroke. iScience, 25 (1), 103539. https://doi.org/10.1016/j.isci.2021.103539”
Allen, T. G. (1997). The ‘sniffer-patch’ technique for detection of neurotransmitter release. Trends Neurosci. 20, 192-197.
Amiaz, R., Kent, I., Rubinstein, K., Sela, B. A., Javitt, D., and Weiser, M. (2015). Safety, tolerability and pharmacokinetics of open label sarcosine added on to anti-psychotic treatment in schizophrenia-preliminary study. Isr. J. Psychiatr. Relat. Sci. 52, 12-15.
Aragon, M. C., Gimenez, C., and Mayor, F. (1987). Stoichiometry of sodium- and chloride-coupled glycine transport in synaptic plasma membrane vesicles derived from rat brain. FEBS Lett. 212, 87-90.
Aubrey, K. R., and Vandenberg, R. J. (2001). N [3-(4′-fluorophenyl)-3-(4′-phenylphenoxy) propyl] sarcosine (NFPS) is a selective persistent inhibitor of glycine transport. Br. J. Pharmacol. 134, 1429-1436.
Aubrey, K. R., Rossi, F. M., Ruivo, R., Alboni, S., Bellenchi, G. C., Le Goff, A., Gasnier, B., and Supplisson, S. (2007). The transporters GlyT2 and VIAAT cooperate to determine the vesicular glycinergic phenotype. J. Neurosci. 27, 6273-6281.
Balkaya, M., Krober, J., Gertz, K., Peruzzaro, S., and Endres, M. (2013). Characterization of long-term functional outcome in a murine model of mild brain ischemia. J. Neurosci. Methods 213, 179-187.
Balu, D. T., Basu, A. C., Corradi, J. P., Cacace, A. M., and Coyle, J. T. (2012). The NMDA receptor co-agonists, D-serine and glycine, regulate neuronal dendritic architecture in the somatosensory cortex. Neurobiol. Dis. 45, 671-682.
Basu, A. C., Tsai, G. E., Ma, C. L., Ehmsen, J. T., Mustafa, A. K., Han, L., Jiang, Z. I., Benneyworth, M. A., Froimowitz, M. P., Lange, N., et al. (2009). Targeted disruption of serine racemase affects glutamatergic neurotransmission and behavior. Mol. Psychiatr. 14, 719-727.
Benedek, A., Moricz, K., Juranyi, Z., Gigler, G., Levay, G., Harsing, L. G., Jr., Matyus, P., Szenasi, G., and Albert, M. (2006). Use of TTC staining for the evaluation of tissue injury in the early phases of reperfusion after focal cerebral ischemia in rats. Brain Res. 1116, 159-165.
Bennett, Rachel E., Robbins, Ashley B., Hu, Miwei, Cao, Xinrui, Betensky, Rebecca A., Clark, Tim, Das, Sudeshna, and Hyman, Bradley T. (2017). Tau induces blood vessel abnormalities and angiogenesis-related gene expression in P301L transgenic mice and human Alzheimer's disease. Proc. Natl. Acad. Sci. USA 115, E1289-E1298.
Benneyworth, M. A., and Coyle, J. T. (2012). Altered acquisition and extinction of amphetamine-paired context conditioning in genetic mouse models of altered NMDA receptor function. Neuropsychopharmacology 37, 2496-2504.
Bergeron, R., Meyer, T. M., Coyle, J. T., and Greene, R. W. (1998). Modulation of N-methyl-_{D}-aspartate receptor function by glycine transport. Proc. Natl. Acad. Sci. USA 95, 15730-15734.
Biegon, A., Liraz-Zaltsman, S., and Shohami, E. (2018). Stimulation of N-methyl-_{D}-aspartate receptors by exogenous and endogenous ligands improves outcome of brain injury. Curr. Opin. Neurol. 31, 687-692.
Biswas, S., and Barma, S. (2020). A large-scale optical microscopy image dataset of potato tuber for deep learning based plant cell assessment. Sci. Data 7, 371.
Bouet, V., Boulouard, M., Toutain, J., Divoux, D., Bernaudin, M., Schumann-Bard, P., and Freret, T. (2009). The adhesive removal test: a sensitive method to assess sensorimotor deficits in mice. Nat. Protoc. 4, 1560-1564.
Caldinelli, L., Pedotti, M., Motteran, L., Molla, G., and Pollegioni, L. (2009). FAD binding in glycine oxidase from Bacillus subtilis. Biochimie 91, 1499-1508.
Chen, Z., Hu, B., Wang, F., Du, L., Huang, B., Li, L., Qi, J., and Wang, X. (2015). Glycine bidirectionally regulates ischemic tolerance via different mechanisms including NR2A-dependent CREB phosphorylation. J. Neurochem. 133, 397-408.
Chen, J., Hu, R., Liao, H., Zhang, Y., Lei, R., Zhang, Z., Zhuang, Y., Wan, Y., Jin, P., Feng, H., and Wan, Q. (2017). A non-ionotropic activity of NMDA receptors contributes to glycine-induced neuroprotection in cerebral ischemia-reperfusion injury. Sci. Rep. 7, 3575.
Chen, Z., Wang, X., Liao, H., Sheng, T., Chen, P., Zhou, H., Pan, Y., Liu, W., and Yao, H. (2020). Glycine attenuates cerebrovascular remodeling via glycine receptor alpha 2 and vascular endothelial growth factor receptor 2 after stroke. Am. J. Transl. Res. 12, 6895-6907.
Choi, J. H., Yu, N. K., Baek, G. C., Bakes, J., Sco, D., Nam, H. J., Baek, S. H., Lim, C. S., Lee, Y. S., and Kaang, B. K. (2014). Optimization of AAV expression cassettes to improve packaging capacity and transgene expression in neurons. Mol. Brain 7, 17.
Cubelos, B., Gimenez, C., and Zafra, F. (2005). Localization of the GLYTI glycine transporter at glutamatergic synapses in the rat brain. Cereb. Cortex 15, 448-459.
Deng, J., Dong, W., Socher, R., Li, L.-J., Li, K., and Fei-Fei, L. (2009). Imagenet: a large-scale hierarchical image database. IEEE Conf. Comput. Vis. Pattern Recogn. 248-255.
Dojo Soeandy, C., Salmasi, F., Latif, M., Elia, A. J., Suo, N. J., and Henderson, J. T. (2019). Endothelin-1-mediated cerebral ischemia in mice: early cellular events and the role of caspase-3. Apoptosis 24, 578-595.
Ehara, A., and Ueda, S. (2009). Application of Fluoro-Jade C in acute and chronic neurodegeneration models: utilities and staining differences. Acta Histochem. Cytochem. 42, 171-179.
Farr, T. D., Liu, L., Colwell, K. L., Whishaw, I. Q., and Metz, G. A. (2006). Bilateral alteration in stepping pattern after unilateral motor cortex injury: a new test strategy for analysis of skilled limb movements in neurological mouse models. J. Neurosci. Methods 153, 104-113.
Forsythe, I. D., Westbrook, G. L., and Mayer, M. L. (1988). Modulation of excitatory synaptic transmission by glycine and zinc in cultures of mouse hippocampal neurons. J. Neurosci. 8, 3733-3741.
Furukawa, H., and Gouaux, E. (2003). Mechanisms of activation, inhibition and specificity: crystal structures of the NMDA receptor NRI ligand-binding core. EMBO J. 22, 2873-2885.
Gibert-Rahola, J., and Villena-Rodriguez, A. (2014). Glutamatergic drugs for schizophrenia treatment. Actas Esp Psiquiatr 42, 234-241.
Gomeza, J., Hulsmann, S., Ohno, K., Eulenburg, V., Szoke, K., Richter, D., and Betz, H. (2003). Inactivation of the glycine transporter 1 gene discloses vital role of glial glycine uptake in glycinergic inhibition. Neuron 40, 785-796.
Gotti, B., Duverger, D., Bertin, J., Carter, C., Dupont, R., Frost, J., Gaudilliere, B., Mackenzie, E. T., Rousseau, J., Scatton, B., et al. (1988). Ifenprodil and SL 82.0715 as cerebral anti-ischemic agents. I. Evidence for efficacy in models of focal cerebral ischemia. J. Pharmacol. Exp. Ther. 247, 1211-1221.
Guastella, J., Brecha, N., Weigmann, C., Lester, H. A., and Davidson, N. (1992). Cloning, expression, and localization of a rat brain high-affinity glycine transporter. Proc. Natl. Acad. Sci. USA 89, 7189-7193.
Han, L., Campanucci, V. A., Cooke, J., and Salter, M. W. (2013). Identification of a single amino acid in GluN1 that is critical for glycine-primed internalization of NMDA receptors. Mol. Brain 6, 36.
Harvey, R. J., and Yee, B. K. (2013). Glycine transporters as novel therapeutic targets in schizophrenia, alcohol dependence and pain. Nat. Rev. Drug Discov. 12, 866-885.
Hatfield, R. H., Mendelow, A. D., Perry, R. H., Alvarez, L. M., and Modha, P. (1991).
Triphenyltetrazolium chloride (TTC) as a marker for ischaemic changes in rat brain following permanent middle cerebral artery occlusion. Neuropathol. Appl. Neurobiol. 17, 61-67.
Herdon, H. J., Godfrey, F. M., Brown, A. M., Coulton, S., Evans, J. R., and Cairns, W. J. (2001). Pharmacological assessment of the role of the glycine transporter GlyT-1 in mediating high-affinity glycine uptake by rat cerebral cortex and cerebellum synaptosomes. Neuropharmacology 41, 88-96.
Hu, R., Chen, J., Lujan, B., Lei, R., Zhang, M., Wang, Z., Liao, M., Li, Z., Wan, Y., Liu, F., et al. (2016). Glycine triggers a non-ionotropic activity of GluN2A-containing NMDA receptors to confer neuroprotection. Sci. Rep. 6, 34459.
Huang, B., Xie, Q., Lu, X., Qian, T., Li, S., Zhu, R., Yu, W., Chen, G., Chen, Z., Xu, X., et al. (2016). GlyT1 inhibitor NFPS exerts neuroprotection via GlyR Alphal subunit in the rat model of transient focal cerebral ischaemia and reperfusion. Cell Physiol. Biochem. 38, 1952-1962.
Ikonomidou, C., and Turski, L. (2002). Why did NMDA receptor antagonists fail clinical trials for stroke and traumatic brain injury? Lancet Neurol. 1, 383-386.
Job, V., Marcone, G. L., Pilone, M. S., and Pollegioni, L. (2002). Glycine oxidase from Bacillus subtilis. Characterization of a new flavoprotein. J. Biol. Chem. 277, 6985-6993.
Johnson, J. W., and Ascher, P. (1987). Glycine potentiates the NMDA response in cultured mouse brain neurons. Nature 325, 529-531.
Johnson, J. W., and Ascher, P. (1992). Equilibrium and kinetic study of glycine action on the N-methyl-_{D}-aspartate receptor in cultured mouse brain neurons. J. Physiol. 455, 339-365.
Kalia, L. V., Kalia, S. K., and Salter, M. W. (2008). NMDA receptors in clinical neurology: excitatory times ahead. Lancet Neurol. 7, 742-755.
Kannangara, T. S., Eadie, B. D., Bostrom, C. A., Morch, K., Brocardo, P. S., and Christie, B. R. (2015). GluN2A^{-/-} mice lack bidirectional synaptic plasticity in the dentate gyrus and perform poorly on spatial pattern separation tasks. Cereb. Cortex 25, 2102-2113.
Ke, M. T., Fujimoto, S., and Imai, T. (2013). SeeDB: a simple and morphology-preserving optical clearing agent for neuronal circuit reconstruction. Nat. Neurosci. 16, 1154-1161.
Kermany, D. S., Goldbaum, M., Cai, W., Valentim, C. C. S., Liang, H., Baxter, S. L., Mckeown, A., Yang, G., Wu, X., Yan, F., et al. (2018). Identifying medical diagnoses and treatable diseases by image-based deep learning. Cell 172, 1122-11231 e9.
Kirchhausen, T., Macia, E., and Pelish, H. E. (2008). Use of dynasore, the small molecule inhibitor of dynamin, in the regulation of endocytosis. Methods Enzymol. 438, 77-93.
Kleckner, N. W., and Dingledine, R. (1988). Requirement for glycine in activation of NMDA-receptors expressed in Xenopus oocytes. Science 241, 835-837.
Kostandy, B. B. (2012). The role of glutamate in neuronal ischemic injury: the role of spark in fire. Neurol. Sci. 33, 223-237.
Lai, M. M., Hong, J. J., Ruggiero, A. M., Burnett, P. E., Slepnev, V. I., De Camilli, P., and Snyder, S. H. (1999). The calcineurin-dynamin I complex as a calcium sensor for synaptic vesicle endocytosis. J. Biol. Chem. 274, 25963-25966.
Lee, C. J., Mannaioni, G., Yuan, H., Woo, D. H., Gingrich, M. B., and Traynelis, S. F. (2007a).
Astrocytic control of synaptic NMDA receptors. J. Physiol. 581, 1057-1081.
Lee, J. K., Park, M. S., Kim, Y. S., Moon, K. S., Joo, S. P., Kim, T. S., Kim, J. H., and Kim, S. H. (2007b). Photochemically induced cerebral ischemia in a mouse model. Surg. Neurol. 67, 620-625.
Lin, C. Y., Liang, S. Y., Chang, Y. C., Ting, S. Y., Kao, C. L., Wu, Y. H., Tsai, G. E., and Lane, H. Y. (2017). Adjunctive sarcosine plus benzoate improved cognitive function in chronic schizophrenia patients with constant clinical symptoms: a randomised, double-blind, placebo-controlled trial. World J. Biol. Psychiatr. 18, 357-368.
Lipton, S. A. (2004). Failures and successes of NMDA receptor antagonists: molecular basis for the use of open-channel blockers like memantine in the treatment of acute and chronic neurologic insults. NeuroRx 1, 101-110.
Liu, X., Smith, B. J., Chen, C., Callegari, E., Becker, S. L., Chen, X., Cianfrogna, J., Doran, A. C., Doran, S. D., Gibbs, J. P., et al. (2005). Use of a physiologically based pharmacokinetic model to study the time to reach brain equilibrium: an experimental analysis of the role of blood-brain barrier permeability, plasma protein binding, and brain tissue binding. J. Pharmacol. Exp. Ther. 313, 1254-1262.
Liu, R., Liao, X. Y., Pan, M. X., Tang, J. C., Chen, S. F., Zhang, Y., Lu, P. X., Lu, L. J., Zou, Y. Y., Qin, X. P., et al. (2019). Glycine exhibits neuroprotective effects in ischemic stroke in rats through the inhibition of MI microglial polarization via the NF-kappaB p65/Hif-1 alpha signaling pathway. J. Immunol. 202, 1704-1714.
Mallorga, P. J., Williams, J. B., Jacobson, M., Marques, R., Chaudhary, A., Conn, P. J., Pettibone, D. J., and Sur, C. (2003). Pharmacology and expression analysis of glycine transporter GlyT1 with [3H]-(N-[3-(4′-fluorophenyl)-3-(4′phenylphenoxy) propyl]) sarcosine. Neuropharmacology 45, 585-593.
Mangin, J. M., Baloul, M., Prado De Carvalho, L., Rogister, B., Rigo, J. M., and Legendre, P. (2003). Kinetic properties of the alpha2 homo-oligomeric glycine receptor impairs a proper synaptic functioning. J. Physiol. 553, 369-386.
Martina, M., Gorfinkel, Y., Halman, S., Lowe, J. A., Periyalwar, P., Schmidt, C. J., and Bergeron, R. (2004). Glycine transporter type 1 blockade changes NMDA receptor-mediated responses and LTP in hippocampal CAI pyramidal cells by altering extracellular glycine levels. J. Physiol. 557, 489-500. Martina, M., B-Turcotte, M. E., Halman, S., Tsai, G., Tiberi, M., Coyle, J. T., and Bergeron, R. (2005). Reduced glycine transporter type 1 expression leads to major changes in glutamatergic neurotransmission of CA1 hippocampal neurones in mice. J. Physiol. 563, 777-793.
Matsumoto, K., Mitani, T. T., Horiguchi, S. A., Kaneshiro, J., Murakami, T. C., Mano, T., Fujishima, H., Konno, A., Watanabe, T. M., Hirai, H., and Ueda, H. R. (2019). Advanced CUBIC tissue clearing for whole-organ cell profiling. Nat. Protoc. 14, 3506-3537.
Metz, G. A., and Whishaw, I. Q. (2009). The ladder rung walking task: a scoring system and its practical application. J. Vis. Exp. 1204.
Molla, G., Motteran, L., Job, V., Pilone, M. S., and Pollegioni, L. (2003). Kinetic mechanisms of glycine oxidase from Bacillus subtilis. Eur. J. Biochem. 270, 1474-1482.
Muller, E., Bakkar, W., Martina, M., Sokolovski, A., Wong, A. Y., Legendre, P., and Bergeron, R. (2013). Vesicular storage of glycine in glutamatergic terminals in mouse hippocampus. Neuroscience 242, 110-127.
Nong, Y., Huang, Y. Q., Ju. W., Kalia, L. V., Ahmadian, G., Wang, Y. T., and Salter, M. W. (2003). Glycine binding primes NMDA receptor internalization. Nature 422, 302-307.
Nong, Y., Huang, Y. Q., and Salter, M. W. (2004). NMDA receptors are movin. Curr. Opin. Neurobiol. 14, 353-361.
Paoletti, P., Neyton, J., and Ascher, P. (1995). Glycine-independent and subunit-specific potentiation of NMDA responses by extracellular Mg2+. Neuron 15, 1109-1120.
Papouin, T., Ladepeche, L., Ruel, J., Sacchi, S., Labasque, M., Hanini, M., Groc, L., Pollegioni, L., Mothet, J. P., and Oliet, S. H. (2012). Synaptic and extrasynaptic NMDA receptors are gated by different endogenous coagonists. Cell 150, 633-646.
Park, C. K., Nehls, D. G., Teasdale, G. M., and McCulloch, J. (1989). Effect of the NMDA antagonist MK-801 on local cerebral blood flow in focal cerebral ischaemia in the rat. J. Cereb. Blood Flow Metab. 9, 617-622.
Pedotti, M., Ghisla, S., Motteran, L., Molla, G., and Pollegioni, L. (2009). Catalytic and redox properties of glycine oxidase from Bacillus subtilis. Biochimie 91, 604-612.
Pineiro, R., Pendlebury, S. T., Smith, S., Flitney, D., Blamire, A. M., Styles, P., and Matthews, P. M. (2000). Relating MRI changes to motor deficit after ischemic stroke by segmentation of functional motor pathways. Stroke 31, 672-679.
Pinto, M. C., Lima, I. V., da Costa, F. L., Rosa, D. V., Mendes-Goulart, V. A., Resende, R. R., Romano-Silva, M. A., de Oliveira, A. C., Gomez, M. V., and Gomez, R. S. (2015). Glycine transporters type 1 inhibitor promotes brain preconditioning against NMDA-induced excitotoxicity. Neuropharmacology 89, 274-281.
Prass, K., and Dirnagl, U. (1998). Glutamate antagonists in therapy of stroke. Restor. Neurol. Neurosci. 13, 3-10.
Qin, X., Akter, F., Qin, L., Xie, Q., Liao, X., Liu, R., Wu, X., Cheng, N., Shao, L., Xiong, X., et al. (2019). MicroRNA-26b/PTEN signaling pathway mediates glycine-induced neuroprotection in SAH injury. Neurochem. Res. 44, 2658-2669.
Rosenmund, C., Stern-Bach, Y., and Stevens, C. F. (1998). The tetrameric structure of a glutamate receptor channel. Science 280, 1596-1599.
Rossi, D. J., Oshima, T., and Attwell, D. (2000). Glutamate release in severe brain ischaemia is mainly by reversed uptake. Nature 403, 316-321.
Rossi, D. J., Brady, J. D., and Mohr, C. (2007). Astrocyte metabolism and signaling during brain ischemia. Nat. Neurosci. 10, 1377-1386.
Sakimura, K., Kutsuwada, T., Ito, I., Manabe, T., Takayama, C., Kushiya, E., Yagi, T., Aizawa, S., Inoue, Y., Sugiyama, H., et al. (1995). Reduced hippocampal LTP and spatial learning in mice lacking NMDA receptor epsilon 1 subunit. Nature 373, 151-155.
Scain, A. L., Le Corronc, H., Allain, A. E., Muller, E., Rigo, J. M., Meyrand, P., Branchereau, P., and Legendre, P. (2010). Glycine release from radial cells modulates the spontaneous activity and its propagation during early spinal cord development. J. Neurosci. 30, 390-403.
Scatton, B. (1994). Excitatory amino acid receptor antagonists: a novel treatment for ischemic cerebrovascular diseases. Life Sci. 55, 2115-2124.
Schallert, T., Fleming, S. M., Leasure, J. L., Tillerson, J. L., and Bland, S. T. (2000). CNS plasticity and assessment of forelimb sensorimotor outcome in unilateral rat models of stroke, cortical ablation, parkinsonism and spinal cord injury. Neuropharmacology 39, 777-787.
Settembre, E. C., Dorrestein, P. C., Park, J. H., Augustine, A. M., Begley, T. P., and Ealick, S. E. (2003). Structural and mechanistic studies on ThiO, a glycine oxidase essential for thiamin biosynthesis in Bacillus subtilis. Biochemistry 42, 2971-2981.
Shahsavar, A., Stohler, P., Bourenkov, G., Zimmermann, I., Siegrist, M., Guba, W., Pinard, E., Sinning, S., Seeger, M. A., Schneider, T. R., et al. (2021). Structural insights into the inhibition of glycine reuptake. Nature 591, 677-681.
Smith, K. E., Borden, L. A., Hartig, P. R., Branchek, T., and Weinshank, R. L. (1992). Cloning and expression of a glycine transporter reveal colocalization with NMDA receptors. Neuron 8, 927-935.
Sommer, Clemens J. (2017). Ischemic stroke: experimental models and reality. Acta Neuropathol. 133, 245-261.
Stanton, P. K., Potter, P. E., Aguilar, J., Decandia, M., and Moskal, J. R. (2009). Neuroprotection by a novel NMDAR functional glycine site partial agonist, GLYX-13. Neuroreport 20, 1193-1197.
Strzelecki, D., Szyburska, J., and Rabe-Jablonska, J. (2014). Two grams of sarcosine in schizophrenia-is it too much? A potential role of glutamate-serotonin interaction. Neuropsychiatr. Dis. Treat. 10, 263-266.
Strzelecki, D., Kaluzynska, O., Szyburska, J., Wlazlo, A., and Wysokinski, A. (2015). No changes of cardiometabolic and body composition parameters after 6-month add-on treatment with sarcosine in patients with schizophrenia. Psychiatr. Res. 230, 200-204.
Toussay, X., Tiberi, M., and Lacoste, B. (2019). Laser doppler flowmetry to study the regulation of cerebral blood flow by G protein-coupled receptors in rodents. Methods Mol. Biol. 1947, 377-387.
Traynelis, S. F., Wollmuth, L. P., McBain, C. J., Menniti, F. S., Vance, K. M., Ogden, K. K., Hansen, K. B., Yuan, H., Myers, S. J., and Dingledine, R. (2010). Glutamate receptor ion channels: structure, regulation, and function. Pharmacol. Rev. 62, 405-496.
Tsai, G., Ralph-Williams, R. J., Martina, M., Bergeron, R., Berger-Sweeney, J., Dunham,
K. S., Jiang, Z., Caine, S. B., and Coyle, J. T. (2004). Gene knockout of glycine transporter 1: characterization of the behavioral phenotype. Proc. Natl. Acad. Sci. USA 101, 8485-8490.
Tsai, P. S., Kaufhold, J. P., Blinder, P., Friedman, B., Drew, P. J., Karten, H. J., Lyden, P. D., and Kleinfeld, D. (2009). Correlations of neuronal and microvascular densities in murine cortex revealed by direct counting and colocalization of nuclei and vessels. J. Neurosci. 29, 14553-14570.
Wang, Y., Jin, K., and Greenberg, D. A. (2007). Neurogenesis associated with endothelin-induced cortical infarction in the mouse. Brain Res. 1167, 118-122.
Wolosker, H., Sheth, K. N., Takahashi, M., Mothet, J. P., Brady, R. O., Jr., Ferris, C. D., and Snyder, S. H. (1999). Purification of serine racemase: biosynthesis of the neuromodulator D-serine. Proc. Natl. Acad. Sci. USA 96, 721-725.
Wu, Q. J., and Tymianski, M. (2018). Targeting NMDA receptors in stroke: new hope in neuroprotection. Mol. Brain 11, 15.
Yamamoto, S., Ohta, H., Abe, K., Kambe, D., Tsukiyama, N., Kawakita, Y., Moriya, M., and Yasuhara, A. (2016). Identification of 1-methyl-N-(propan-2-yl)-N-({2-[4-(trifluoromethoxy)phenyl]pyridin-4-yl}methyl)-1
H-imidazole-4-carboxamide as a potent and orally available Glycine transporter 1 inhibitor. Chem. Pharm. Bull. (Tokyo) 64, 1630-1640.
Yao, W., Ji, F., Chen, Z., Zhang, N., Ren, S. Q., Zhang, X. Y., Liu, S. Y., and Lu, W. (2012). Glycine exerts dual roles in ischemic injury through distinct mechanisms. Stroke 43, 2212-2220.
Yu, C., Wang, J., Peng, C., Gao, C., Yu, G., and Sang, N. (2018). Bisenet: bilateral segmentation network for real-time semantic segmentation. Proc. Eur. Conf. Comput. Vis. (Eccv), 325-341.
Zhao, D., Chen, J., Zhang, Y., Liao, H. B., Zhang, Z. F., Zhuang, Y., Pan, M. X., Tang, J. C., Liu, R., Lei, Y., et al. (2018). Glycine confers neuroprotection through PTEN/AKT signal pathway in experimental intracerebral hemorrhage. Biochem. Biophys. Res. Commun. 501, 85-91.
Zheng, C., Qiao, Z. H., Hou, M. Z., Liu, N. N., Fu, B., Ding, R., Li, Y. Y., Wei, L. P., Liu, A. L., and Shen, H. (2017). GLYX-13, a NMDA receptor glycine-site functional partial agonist, attenuates cerebral ischemia injury in vivo and vitro by differential modulations of NMDA receptors subunit components at different post-ischemia stage in mice. Front. Aging Neurosci. 9, 186.

Compounds of the class of glycine transporter-1 antagonist compounds have been used for many years in clinical trials attempting to treat conditions such as, for example, Schizophrenia, Schizoaffective disorder, and Alzheimer's, and have been proven to be safe for use in humans, even with long term daily use.

A selection of pre-clinical and clinical trials is listed hereinbelow for the glycine transporter-1 antagonists: Bitopertin “RG1678” (Roche); BI 425809 (Boehringer); Sarcosine; and PF-03463275 (Pfizer).

### Bitopertin

### Pre-Clinical

Armbruster, A., Neumann, E., Kötter, V., Hermanns, H., Werdehausen, R., & Eulenburg, V. (2018). The glytl inhibitor bitopertin ameliorates allodynia and Hyperalgesia in animal models of neuropathic and inflammatory pain. Frontiers in Molecular Neuroscience, 10. https://doi.org/10.3389/fnmol.2017.00438
Borroni, E., Zhou, Y., Ostrowitzki, S., Alberati, D., Kumar, A., Hainzl, D., . . . . Wong, D. F. (2013). Pre-clinical characterization of [11C] R05013853 as a novel Radiotracer for imaging of the glycine transporter type 1 by positron emission tomography. NeuroImage, 75, 291-300. doi: 10.1016/j.neuroimage.2011.11.090.
Eddins, D., Hamill, T. G., Puri, V., Cannon, C. E., Vivian, J. A., Sanabria-Bohorquez, S. M., . . . . Uslaner, J. M. (2013). The relationship between glycine transporter 1 occupancy and the effects of the glycine transporter 1 inhibitor RG1678 or ORG25935 on object retrieval performance in scopolamine impaired rhesus monkey. Psychopharmacology, 231 (3), 511-519. doi: 10.1007/s00213-013-3260-0

### Clinical Phase 1

Martin-Facklam, M., Pizzagalli, F., Zhou, Y., Ostrowitzki, S., Raymont, V., Braši?, J. R., . . . . Wong, D. F. (2012). Glycine transporter type 1 occupancy by Bitopertin: A positron emission tomography study in healthy volunteers. Neuropsychopharmacology, 38 (3), 504-512. doi: 10.1038/npp.2012.212

### Clinical Phase 2

Umbricht, D., Alberati, D., Martin-Facklam, M., Borroni, E., Youssef, E. A., Ostland, M., . . . . Santarelli, L. (2014). Effect of Bitopertin, a glycine reuptake inhibitor, on negative symptoms of schizophrenia. JAMA Psychiatry, 71 (6), 637. doi: 10.1001/jamapsychiatry.2014.163

### Clinical Phase 3

Pinard, F., Borroni, E., Koerner, A., Umbricht, D., & Alberati, D. (2018). Glycine transporter type I (glyt1) inhibitor, Bitopertin: A journey from lab to patient. CHIMIA, 72 (7-8), 477. doi: 10.2533/chimia.2018.477

### BI 425809

### Pre-clinical

Rosenbrock, H., Dorner-Ciossek, C., Giovannini, R., Schmid, B., & Schuelert, N. (2022). Effects of the glycine transporter-1 inhibitor Iclepertin (BI 425809) on sensory processing, neural network function, and cognition in animal models related to schizophrenia. Journal of Pharmacology and Experimental Therapeutics, 382 (2), 223-232. doi: 10.1124/jpet. 121.001071

### Pre-clinical & Clinical

Rosenbrock, H., Desch, M., Kleiner, O., Dorner-Ciossec, C., Schmid, B., Keller, S., . . . . Wind, S. (2018). Evaluation of Pharmacokinetics and Pharmacodynamics of BI 425809, a Novel GlyT1 Inhibitor: Translational Studies. Clin Transl Sci (2018) 11, 616-623; doi: 10.1111/cts.12578

### Clinical Phase 1

Moschetti, V., Schlecker, C., Wind, S., Goetz, S., Schmitt, H., Schultz, A., . . . . Desch, M. (2018). Multiple rising doses of oral bi 425809, a Glyt1 inhibitor, in young and elderly healthy volunteers: A randomised, double-blind, phase I study investigating safety and pharmacokinetics. Clinical Drug Investigation, 38 (8), 737-750. doi: 10.1007/s40261-018-0660-2
Moschetti et al. (2018). Safety, Tolerability and Pharmacokinetics of Oral BI 425809, a Glycine Transporter 1 Inhibitor, in Healthy Male Volunteers: A Partially Randomised, Single-Blind, Placebo-Controlled, First-in-Human Study. ClinicalTrials.gov Identifier: NCT02068690

### Clinical Phase 2

Harvey et al. (2020). Evaluation of the Efficacy of BI 425809 Pharmacotherapy in Patients with Schizophrenia Receiving Computerized Cognitive Training: Methodology for a Double-Blind, Randomized, Parallel-Group Trial. ClinicalTrials.gov Identifier: NCT03859973

### Clinical Phase 3

CONNEX-1. ClinicalTrials.gov Identifier: NCT04846868
CONNEX-2. ClinicalTrials.gov Identifier: NCT04846881
CONNEX-3. ClinicalTrials.gov Identifier: NCT04860830
CONNEX-4. ClinicalTrials.gov Identifier: NCT05211947

### Sarcosine

### Pre-Clinical.

Pinto, M., Simão, F., Da Costa, F., Rosa, D., De Paiva, M., Resende, R., . . . . Gomez, R. (2014). Sarcosine preconditioning induces ischemic tolerance against global cerebral ischemia. Neuroscience, 271, 160-169. doi: 10.1016/j.neuroscience.2014.04.054

### Clinical Phase 2

Tsai, G., Lane, H., Yang, P., Chong, M., & Lange, N. (2004). Glycine transporter I inhibitor, N-methylglycine (sarcosine), added to antipsychotics for the treatment of schizophrenia. Biological Psychiatry, 55 (5), 452-456. doi: 10.1016/j.biopsych.2003.09.012
Tsai, C., Huang, H., Liu, B., Li, C., Lu, M., Chen, X., . . . . Lane, H. (2014). Activation of N-methyl-_{D}-aspartate receptor glycine site temporally ameliorates neuropsychiatric symptoms of parkinson's disease with dementia. Psychiatry and Clinical Neurosciences, 68 (9), 692-700. doi: 10.1111/pcn.12175

### PF-03463275

### Pre-Clinical

Liu, C., Pettersen, B., Seitis, G., Osgood, S., & Somps, C. (2014). Glyt1 inhibitor reduces oscillatory potentials of the electroretinogram in rats. Cutaneous and Ocular Toxicology, 33 (3), 206-211. doi: 10.3109/15569527.2013.833937
Roberts, B. M., Shaffer, C. L., Seymour, P. A., Schmidt, C. J., Williams, G. V., & Castner, S. A. (2010). Glycine transporter inhibition reverses ketamine-induced working memory deficits. NeuroReport, 21 (5), 390-394. doi: 10.1097/wnr.0b013e3283381a4e

### Clinical Phase 1

Pfizer. (2008). A Phase 1B Inpatient, Randomized, Double-Blind, Placebo-Controlled, Crossover Study Of The Safety And Efficacy Of Two Fixed Doses Of PF-03463275 In Adjunctive Treatment Of Cognitive Deficits In Schizophrenia. ClinicalTrials.gov Identifier: NCT00567203
Pfizer. (2010). Pharmacokinetics, Safety and Tolerability Study of PF-03463275 in Healthy Male Japanese and Western Subjects. ClinicalTrials.gov Identifier: NCT01159626

### Clinical Phase 2

D'Souza, D. C., Carson, R. E., Driesen, N., Johannesen, J., Ranganathan, M., Krystal, J. H., . . . . Pittman, B. (2018). Dose-related target occupancy and effects on circuitry, behavior, and neuroplasticity of the glycine transporter-1 inhibitor PF-03463275 in healthy and schizophrenia subjects. Biological Psychiatry, 84 (6), 413-421. doi: 10.1016/j.biopsych.2017.12.019
Pfizer. (2009-2012). A Randomized Phase 2, Double-Blind, Placebo-Controlled, Multi-Center Study Of PF-03463275 As Add-On Therapy In Outpatients With Persistent Negative Symptoms Of Schizophrenia Treated With A Stable Dose Of A Second Generation Antipsychotic. ClinicalTrials.gov Identifier: NCT00977522
Pfizer. (2013-2021). Translational Neuroscience Optimization of GlyT1 Inhibitor. ClinicalTrials.gov Identifier: NCT01911676

### Efficacy

In light of the above it is soundly predicted that administering a therapeutically effective amount of a glycine transporter-1 antagonist compound to a patient as vascular dementia and/or stroke prophylactic/treatment agent according to the invention will effect neuro protection in a human patient's brain by sufficiently increasing the level of glycine in the brain for blocking the NDMA receptors.

As evidenced hereinabove, it has been demonstrated, in vivo, that elevation of extracellular glycine by administering a GlyT-1 antagonist compound resulted in a decreased stroke volume and an attenuation of motor deficits in mice following ischemic stroke induced by PT or ET-1.

This was observed when the GlyT-1 antagonist compound was administered pre-stroke, or post-stroke, thus demonstrating efficacy of the GlyT-1 antagonist compound as a stroke prophylactic agent as well as a stroke treatment agent. Considering the fact that mice have similar NDMA receptors as humans, it is soundly predicted that administering a GlyT-1 antagonist compound to humans pre-stroke or post-stroke will also result in neuro protection.

GlyT-1 antagonist compounds have been used for decades for treating primarily Schizophrenia, Depression, and to lesser extent Dementia with numerous human trials being conducted for different GlyT-1 antagonist compounds. The treatment of Schizophrenia, Depression, and Dementia is based on the same process of blocking the NDMA receptors using an increased level of glycine.

The only difference between the use of GlyT-1 antagonist compounds for treating Schizophrenia, Dementia, and Depression and for treating ischemic stroke is that the NMDA receptors work less than they should in case of Schizophrenia, Depression, and Dementia and glycine is used for “excitation” of the NMDA receptors while in case of ischemic stroke NMDA receptors work more than they should and glycine is used for “inhibition”.

The doses of 4 example GlyT-1 antagonist compounds used in clinical trials, a selection thereof is listed hereinabove, were in a consistent range between 5 mg and 25 mg with the most frequently used dose being 10 mg taken as a tablet daily over several weeks. It has been demonstrated, Rosenbrock et al., 2018 and Moschetti et al., 2018, that these doses provide a sufficiently high level of glycine in the human brain to block at least 50% of the NDMA receptors, which has been found as being optimal and a further increase in dose does not increase efficacy but causes very mild side effects.

Considering that it has been demonstrated, in vivo, that elevation of extracellular glycine by administering a GlyT-1 antagonist compound pre-stroke or post-stroke resulted in a decreased stroke volume and an attenuation of motor deficits in mice following an induced ischemic stroke, and that in clinical trials administration of GlyT-1 antagonist compounds in a dosing range between 5 mg and 25 mg provide a sufficiently high level of glycine in the human brain to block at least 50% of the NDMA receptors, a sound prediction can be made that administration of a GlyT-1 antagonist compound to a human in a dosing range between 5 mg and 25 mg, pre-stroke as a prophylactic agent or post-stroke as a treatment agent, will effect neuro protection in humans.

### Safety

The doses of 4 example GlyT-1 antagonist compounds used in clinical trials, a selection thereof is listed hereinabove, were in a consistent range between 5 mg and 25 mg typically taken as a tablet daily over long term and was usually very well tolerated. Even high doses such as, for example, 175 mg for Bitopertin “RG1678” and 2 g for Sarcosine, were still relatively well tolerated. Therefore, a sound prediction can be made that administration of a GlyT-1 antagonist compound to a human in a dosing range between 5 mg and 25 mg for use as a vascular dementia and/or stroke prophylactic/treatment agent will be safe even with long term daily use in humans.

Dosages may be varied to optimize a therapeutic and/or prophylactic regimen in a particular subject or group of subjects. A therapeutically effective dose in accordance with the present invention may depend upon the age of the subject, the gender of the subject, the particular pathology, the severity of the symptoms, and the general state of the subject's health.

The single, double, triple, and quadruple asterisks refer to the annotation in the corresponding figures.

The present invention has been described herein with regard to preferred embodiments. However, it will be obvious to persons skilled in the art that a number of variations and modifications can be made without departing from the scope of the invention as described herein.

The embodiments of the invention in which an exclusive property or privilege is claimed are defined as follows:

1. Use of a compound of the class of glycine transporter-1 antagonist compounds as a vascular dementia prophylactic agent.

2. Use of a compound of the class of glycine transporter-1 antagonist compounds as a stroke prophylactic agent.

3. Use of a compound of the class of glycine transporter-1 antagonist compounds as a vascular dementia treatment agent.

4. Use of a compound of the class of glycine transporter-1 antagonist compounds as a stroke treatment agent.
